(19) United States
(12) Patent Application Publication
(10) Pub. No.: US 2025/0122251 A1
(43) Pub. Date: Apr. 17, 2025

(54) EXPRESSION IN MAMMALIAN CELLS WITH SIGNAL PEPTIDE

(71) Applicant: DAPCEL, Inc., Cleveland, OH (US)

(72) Inventor: Jennet Orazmuradovna Beesley, Runcorn (GB)

(21) Appl. No.: 18/939,084

(22) Filed: Nov. 6, 2024

Related U.S. Application Data
(62) Division of application No. 15/769,180, filed on Apr. 18, 2018, now Pat. No. 12,173,036.

(30) Foreign Application Priority Data
Oct. 23, 2015 (GB) 1518792.5

(51) Int. Cl. C07K 14/435; C12N 15/62; C12N 15/85; C12P 21/02; C12Q 1/66

(52) U.S. Cl. CPC C07K 14/43509; C12N 15/625; C12N 15/85; C12Q 1/66; C07K 2319/02; C12P 21/02

(57) ABSTRACT

The present invention relates to in vitro expression of proteins and particularly, although not exclusively, to expression of proteins in mammalian cell lines. In particular, the present invention relates to the provision of a novel vector for protein expression, and methods of using such vector in the expression of proteins in mammalian cell lines.

### CROSS-REFERENCE TO RELATED APPLICATIONS

This application is a divisional of U.S. patent application Ser. No. 15/769,180, filed Apr. 18, 2018, now U.S. Pat. No. 12,173,036, which is a 371 of PCT Patent Application Serial No. PCT/EP2016/075415 filed Oct. 21, 2016, which claims priority to Great Britain Patent Application Serial No. 1518792.5 filed Oct. 23, 2015, the entire contents of which are incorporated herein by reference.

### INCORPORATION BY REFERENCE STATEMENT

This application incorporates by reference a sequence listing submitted herewith as dapc200002 us02_seq_listing.xml, created on Oct. 28, 2024, and having a file size of 128,118 bytes.

### FIELD OF THE INVENTION

The present invention relates to in vitro expression of proteins and particularly, although not exclusively, to expression of proteins in mammalian cell lines. In particular, the present invention relates to the provision of a novel vector for protein expression, and methods of using such vector in the expression of proteins in mammalian cell lines.

### BACKGROUND TO THE INVENTION

According to MarketResearch.com, a supplier of the latest in independent medical market research in diagnostics, biotech, pharmaceuticals, medical devices and healthcare, the biopharmaceutical and vaccine production market was estimated to reach $41 billion in 2014. Despite the recent economic crisis, the demand for biologics production is on the increase, as it has been for the last almost 3 decades. Mammalian cell culture occupies a central role in supplying biotech drug products and vaccines targeting various diseases.

Proteins produced in the mammalian cells are very important not only as therapeutics with least side effects due to their identical chemical and physical properties to their natural prototypes, but also for the production of drug targets. While the microbial expression platform is more economical in terms of overall yield, only the mammalian system can deliver the best qualities of the expected product: proper glycosylation, protein folding and, as a result, full function.

Protein expression and purification is not a routine process and still remains somewhat of an art, predicated on valuable skillsets of technicians. It can also be very restrictive and its success is strongly dictated by the quality of the personnel, their relevant practical experiences and availability of advanced instrumentation.

The progress achieved in terms of driving up mammalian recombinant yields is astonishing: from a few micrograms to a few grams per litre. This has been achieved by extensive research and development work aimed at the several bottlenecks in the production process. They have included creation of genetically engineered fine-tuned cell lines, improved media formulation and design of very efficient expression vectors. However, this research and development work remains costly and sustainable by big companies only.

Academic set up occupies the other end of the scale for recombinant protein production. While it does not require industrial amounts of proteins, it often deals with difficult targets which need to be produced on a limited budget. Also, even at a smaller scale, protein production faces one of the main challenges when compared with E. coli expression-low yield.

### SUMMARY OF THE INVENTION

The present invention provides a nucleic acid expression vector for expressing a peptide in a mammalian cell, the vector comprising an expression cassette;
the expression cassette comprising:
   a promoter;
   nucleic acid encoding signal peptide from Gaussia luciferase;
   a multiple cloning site, for inserting nucleic acid encoding the peptide in-frame with the signal peptide; and
   a 3′ UTR sequence.

Preferably, the nucleic acid expression vector consists of 6000 or fewer nucleic acid base pairs, or more preferably 5000 or fewer nucleic acid base pairs.

At it's most general, the invention provides a nucleic acid expression vector having fewer than 5000 base pairs, and comprising a Gaussia signal peptide sequence. The nucleic acid expression vector may additionally comprise a selectable marker, expressed under the control of a weak promoter.

In some cases, the vector also comprises a selectable marker. The selectable marker may be arranged downstream of a weak promoter. The nucleic acid encoding the signal peptide from Gaussia luciferase (GLUC) may encode a peptide having at least 90% sequence identity to SEQ ID NO: 1. The expression cassette may further comprise nucleic acid encoding the peptide of interest, such as a mammalian protein.

Expression of the gene of interest is under the control of a promoter. The promotor may be a promoter for constitutive expression, such as a CMV promoter. The promoter may be a promoter for inducible expression, such as a doxycycline inducible promoter or a riboswitch.

The expression cassette may additionally comprise a translational enhancer, such as SP163. The expression cassette may further comprise nucleic acid encoding a tag, such as a SUMO-tag.

In some cases, the vector is smaller than 5.5 Kb, preferably smaller than 5 kb.

In another aspect, the invention provides a host cell comprising a nucleic acid expression vector. The host cell may be a mammalian cell, such as a HEK293 cell, a CHO cell, a COS cell, a HeLa cell, a Vero cell, a NSO cell, a Jurkat cell, a BHK cell, an MCF cell, or an L cell mouse fibroblast (such as the cell line deposited as ATCC CRL-2648).

In a further aspect, the invention provides a nucleic acid expression vector comprising an expression cassette, the expression cassette comprising:
a promoter;
nucleic acid encoding Gaussia luciferase signal peptide;
a multiple cloning site, for inserting nucleic acid encoding a polypeptide of interest in-frame with the Gaussia luciferase signal peptide; and
a polyadenylation sequence;
the nucleic acid expression vector further comprising nucleic acid encoding a selectable marker downstream of an SV40 promoter, or a promoter that has weaker expression induction than an SV40 promoter.

Also provided are kits, such as kit for gene expression comprising a vector according to any one of claims, a mammalian cell, and a reagent.

In a further aspect, the invention provides methods. Methods include methods of making a nucleic acid expression vector, comprising introducing a Gaussia signal peptide into a vector, such as a pPEF vector. Such methods result in a nucleic acid expression vector that is smaller than 5000 base pairs in length.

Certain aspects disclosed herein provide methods for producing a protein. Some methods disclosed herein comprise inserting nucleic acid encoding the gene of interest into a nucleic acid expression vector according to the invention, transfecting the nucleic acid expression vector comprising the gene of interest into a mammalian cell; and culturing the transfected cell. Methods disclosed herein may also involve a step of purifying the protein encoded by the gene of interest.

In an aspect of the invention, there is provided a vector having the sequence set out in any one of FIG. 19, 20, 21, 22, 23, 24, 25 or 26. Preferably, the vector has a sequence as shown in any one of FIGS. 19, 20, 25, 26. In some cases, the vector has a sequence of at least 80% sequence identity to the nucleic acid sequence set out in one of FIG. 19, 20, 21, 22, 23, 24, 25 or 26. More preferably, the sequence is about 85%, 90%, 95%, 97%, 98%, 99% or 100% identical to the nucleic acid sequence set out in one of FIG. 19, 20, 21, 22, 23, 24, 25 or 26.

The vector may comprise a degree of sequence identity to a sequence set out in on of FIG. 19, 20, 21, 22, 23, 24, 25 or 26, and additionally include one or more elements selected from an expression cassette, promotor, signal peptide, multiple cloning site, 3′UTR, and selectable marker under the control of a weak promoter. In addition to having the stated degree of sequence identity, the vector may be smaller than 5.5 kb, smaller than 5. kb, or smaller than 4.5 kb.

In certain aspects disclosed herein, the vector does not comprise a sequence encoding F1ori, or a sequence having at least 80%, 85%, 90%, 95% or 100% sequence identity to a sequence encoding F1ori, or does not comprise a sequence encoding EM7, or a sequence having at least 80%, 85%, 90%, 95% or 100% sequence identity to a sequence encoding EM7.

An alternative embodiment of the present invention provides a nucleic acid expression vector for expressing a peptide in a mammalian cell, the vector comprising an expression cassette;
the expression cassette comprising:
   a promoter;
   nucleic acid encoding signal peptide from PDX-Furin;
   a multiple cloning site, for inserting nucleic acid encoding the peptide in-frame with the signal peptide; and
   a 3′ UTR sequence;

Preferably, the nucleic acid expression vector consists of 6000 or fewer nucleic acid base pairs.

Also provided herein is a modified EF1A promotor. The modified EF1A promotor may be used in the vectors described herein to drive expression of the selectable marker. Modified EF1A promotor may have the sequence of “minimal version of EF1A1” or “minimal version of EF1A2” as set out in FIG. 37, or a sequence having at least 90%, 91%, 92%, 93%, 94%, 95%, 96%, 97% 98%, or 99% sequence identity to the sequence set out in FIG. 37. The modified EF1A promoter described herein has a reduced size as compared to native EF1A promotor. In some aspects, the modified EF1A promotor described herein contains fewer than 350 bp, 330 bp, 310 bp, 300 bp, 290 bp, 280 bp, 270 bp, 260 bp, 250 bp, 240 bp, 230 bp, 220 bp, 210 bp, 200 bp, 190 bp, 180 bp, 170 bp, 160 bp, 150 bp, or 140 bp. Preferably, the modified EF1A promotor described herein contains fewer than 200 bp. The modified EF1A promoter preferably has attenuated activity as compared to the native EF1A sequence.

In some aspects, the invention is vector pPEF5PuroR, having a sequence as set out in FIGS. 31A-31B, or having 80%, 85%, 90%, 95% or 98% sequence identity to the sequence set out in FIGS. 31A-31B.

In one preferred arrangement, the vector is pPEF5PuroR deposited by University of Manchester on 18 Oct. 2016 at PHE Culture Collections (Public Health England, Porton Down, Salisbury, SP4 0JG, UK) under Provisional accession number 16101801 in accordance with the provisions of the Budapest Treaty. This is the European Collection of Authenticated Cell Culture (ECACC).

### BRIEF DESCRIPTION OF THE FIGURES

Embodiments and experiments illustrating the principles of the invention will now be discussed with reference to the accompanying figures in which:

FIG. 1. Differential Expression of a1-PDX in pcDNA™4/HisMax vector, pCEP-Pu and RTS-1. Conditioned serum-free concentrated medium from: Lane 1: non-transfected HEK293EBNA1; Lane 2: HEK293EBNA1 stably expressing pCEP-Pu:PLA2R; Lane 3: HEK293EBNA1 stably expressing pCEP-Pu:PLA2R and pcDNA™4/HisMax: a1 vector; Lane 4:HEK293EBNA1 stably expressing pCEP-Pu:PLA2R and transiently expressing pCEP-Pu: a1 PDX; Lane 5:HEK293EBNA1 stably expressing pCEP-Pu:PLA2R and pRTS-1: a1 PDX induced with 0.5 μg/ml doxycylcine.

FIGS. 2A-2B. Improving the yield and secretion of recombinant proteins through vector modifications. FIG. 2A: pCEP-Pu:AC7 (Kohfeldt et al 1997) based on pCEP4 (Invitrogen)—the most popular vector for episomal expression; FIG. 2B: pcDNA™4/HisMax (Invitrogen) vector, an advanced version of pcDNA™3.1 vector from the same manufacturer—the most popular vector for stable expression. Transcription enhancer—SP163 from pcDNA™4/HisMax vector (Invitrogen).

FIGS. 3A-3E. Synthetic DNA fragments. FIG. 3A. Fragment 1 (SEQ ID NO:4) with SP163 enhancer (capitalised, underlined), T7 promoter/priming site (capitalised, italics), BM40 signal peptide, KpnI at the 5′ end (capitalised, bold), NheI at the 3′ end (lower case, bold) and silenced internal NheI site (enlarged, capitalised, italicised)-298 bp; FIG. 3B. Fragment 2 (SEQ ID NO:5) with SP163 enhancer (capitalised, underlined), T7 promoter/priming site (capitalised, italics), Gaussia signal peptide, KpnI at the 5′ end (capitalised, bold), NheI at the 3′ end (lower case, bold) and silenced internal NheI site (enlarged, capitalised, italicised)—301 bp; FIG. 3C. Fragment 3 (SEQ ID NO:6) with T7 promoter/priming site (capitalised, italics), Gaussia signal peptide, KpnI at the 5′ end (capitalised, bold), NheI at the 3′ end and (lower case, bold) silenced internal BamHI site (enlarged and italicised)—116 bp FIG. 3D. F1ori sequence (SEQ ID NO: 7); FIG. 3E. EM7 sequence (SEQ ID NO:8)

FIG. 4. Differential expression of sVAP-1 in the modified vectors. Yield in pCEP-Pu-up to 60 mg/L (Heuts et al, 2011); Yield in pPEF4-100 mg/L (predicted); Yield reported in literature-0.93 mg/L (Ohman et al, 2006). * indicates a combination which has been routinely tested in the laboratory and was used here as a control.

FIGS. 5A-5B. Differential expression of PLA2R1 in the modified vectors. FIG. 5A: Coomassie Staining; FIG. 5B: Western blot; Yield in pCEP-Pu-20-200 μg/ml; Yield in pPEF2-6 mg/L. * indicates a combination which has been routinely tested in the laboratory and was used here as a control.

FIG. 6A. Clustal alignment of our minimal (pPEF) (SEQ ID NO:10), commercial (based on the DNA sequence provided by Oxford Genetics) (SEQ ID NO:11) and published chromosomal sequence of EF1A promoter (GenBank: J04617.1) (SEQ ID NO:9) Part 1;

FIG. 6B. Clustal alignment part 2. FIG. 6C. Synthetic fragment I with the minimal EF1A (1206 bp) SEQ ID NO: 12); FIG. 6D. Synthetic fragment II with the SV40 promoter (1214 bp) (SEQ ID NO:13).

FIGS. 7A-7B. Comparison of human EF1A promoter with SV40 for FIG. 7A. sVAP expression in pPEF5.1 vector and FIG. 7B. PLA2R expression in pPEF5.1Max vector. pPEF5.1 vector contains antibiotic resistance gene under minimal EF1-alpha promoter (4× smaller than the commercial version); pPEF5.2 and pPEF5.2Max contain antibiotic resistance gene under SV40 promoter/origin of replication;

FIG. 8. Expression of HPSE2 1. Conditioned serum-free medium from HEK293 cells stably expressing pcDNA™3.1:HPSE2_FL vector; 2. Cell lysate of HEK293 cells stably expressing pcDNA™3.1:HPSE2_FL vector; 3. Conditioned serum-free medium from HEK293 EBNA1 cells stably expressing pPEF4:HPSE2_A41; 4. Cell lysate of HEK293EBNA1 cells stably expressing pPEF4:HPSE2_A41; 5. Conditioned serum-free medium from HEK293EBNA1 cells stably expressing pPEF4:HPSE2_FL; 6. Cell lysate of HEK293EBNA1 cells stably expressing pPEF4:HPSE2_FL. No published yield to compare to. pPEF4 and pcDNA™3.1 vectors share high identity in terms of vector backbone sequence similarity.

FIGS. 9A-9C. Expression of Sumo-Noggin in pPEF5.2 and pPEF5.2Max. FIG. 9A. Coomassie staining and FIG. 9B. western blot analysis of conditioned serum free medium from 1. Non-transfected HEK293EBNA1 cells (negative control); 2. pPEF5.2 Sumo-Noggin cells, 5 day harvest; and 3. pPEF5.2/Max Sumo-Noggin cells, 5-day harvest; FIG. 9C. Fusion protein made up of 10× His tag, N-terminal SUMOstar secretory tag (LifeSensors) and the mature form of Noggin aa 28-232 (underlined) (SEQ ID NO:14).

FIG. 10. Sumo-Noggin digest and recovery of Noggin. 1. Protein size marker; 2. Initial input (pooled eluted fractions of Ni-purified Sumo-Noggin); 3. SUMOstar digest at 30° C. for 1 hour (recommended by manufacturer); 4. SUMOstar digest at 40° C. for ⅕ hour (optimal conditions); 5. Noggin recovery after digest.

FIGS. 11A-11B. Expression of RET (a transmembrane domain containing protein). FIG. 11A. Published data of Myers S M and Mulligan L M (Cancer Res. 2004:64:4453-4463. RET expression in HEK293EBNA1 cells stably expressing RET in pCEP derivative vector. RET protein was immunoprecipitated from lysed cells with isoform specific antisera and immunoblotted with the same antisera; and FIG. 11B. Western blotting of pPEF5.1 Max; lane 1: cell lysate of Non-transfected HEK 293 EBNA cells; Lane 2 cell lysate of HEK293 EBNA 1 cells stably expressing RET in pPEF5.1Max.

FIGS. 12A-12B. The induction of A105 MMP-12 expression in HEK293 parental and HEK293 EBNA1 cell lines. Western blotting with anti-MMP12 (hemopexin domain). FIG. 12A. HEK293EBNA1 cells; FIG. 12B: HEK293 parental cells.

FIG. 13. Expression of MMP-12 proprotein and the effect of MMP inhibitor. Western blotting with anti-MMP12 (catalytic domain); 1. Conditioned medium from nontransfected parental HEK293EBNA1 cells (negative control); 2. Conditioned medium harvested from MMP12 pro-form HEK293 EBNA1 cells (positive control); 3-5 conditioned media from the inhibitor-treated MMP12 pro-form HEK293 EBBA1 cells.

FIG. 14. Cloning of Ricin-C3 P2A of PLA2R1 (SEQ ID NO: 15). Ricin (aa 20-173) C3 (aa 516-660) domains with P2A bicistronic motif (underlined) and engineered Furin cleavage site (enlarged, bold, italicised).

FIG. 15. Purification of Ricin domain of PLA2R1. 1. Protein size marker; 2. Conditioned serum-free medium from Ricin C3P2A cells, 7 day harvest input; 3. Unbound flow-through; 4. Wash; 5. Eluate.

FIG. 16A. pPEF2 vector carrying elements of pCEP-Pu/AC7 and pcDNA™4/HisMax vector; FIG. 16B. pPEF4 vector corresponding to pPEF2 vector but lacking SP16 Transcriptional enhancer; FIG. 16C. pPEF5.2 vector corresponding to the pPEF4 vector but lacking f1 origin and EM-7 promoter. FIG. 16D. pPEF5.2Max vector as pPEF2 but without f1 origin and EM1 promoter; FIG. 16E. pPEF1 vector; FIG. 16F. pPEF3 vector;

FIG. 16G. pPEF5.1 vector.

FIG. 17. pPEF5.2 (SEQ ID NO: 16) Multiple cloning site

FIG. 18. pPEF5.2 annotations table

FIGS. 19A-19B. pPEF5.2 sequence (SEQ ID NO: 17), FIG. 19A. nucleic acids 1 to 2750; FIG. 19B. nucleic acids 2751 to 4561.

FIGS. 20A-20B. Ppef5.2Max sequence (SEQ ID NO: 18). FIG. 20A. nucleic acids 1 to 2820; FIG. 20B nucleic acids 2821 to 4746.

FIGS. 21A-21B. pPEF1 sequence (SEQ ID NO: 19). FIG. 21A. nucleic acids 1 to 2940; FIG. 21B: nucleic acids 2941 to 5223.

FIGS. 22A-22B. pPEF2 sequence (SEQ ID NO:20). FIG. 22A. nucleic acids 1 to 3060; FIG. 22B: nucleic acids 3061 to 5226.

FIGS. 23A-23B. pPEF3 sequence SEQ ID NO:21). FIG. 23A. nucleic acids 1 to 3060; FIG. 23B: nucleic acids 3061 to 5068.

FIGS. 24A-24B. pPEF4 sequence (SEQ ID NO:22). FIG. 24A. nucleic acids 1 to 3060; FIG. 24B: nucleic acids 3061 to 5041.

FIGS. 25A-25B. pPEF5.1 sequence (SEQ ID NO:23). FIG. 25A. nucleic acids 1 to 3060; FIG. 25B: nucleic acids 3061 to 4553.

FIGS. 26A-26B. pPEF5.1Max sequence (SEQ ID NO:24). FIG. 26A. nucleic acids 1 to 3060; FIG. 26B: nucleic acids 3061 to 4738.

FIGS. 27A-27C. pCEP-Pu/AC7 sequence (SEQ ID NO:25). FIG. 27A. nucleic acids 1 to 3180; FIG. 27B: nucleic acids 3181 to 6540; FIG. 27C: nucleic acids 6541 to 9901.

FIGS. 28A-281. Sequences of vector elements. FIG. 28A: QBI SP163 translational enhancer (SEQ ID NO:26). FIG. 28B. Polylinker/MCS with Lumio and 10×His tags embedded (SEQ ID NO:27) (SEQ ID NO:28); FIG. 28C: Polylinker/MCS without Lumio and 10×His tag (SEQ ID NO:29) (SEQ ID NO:30); FIG. 28D: a1-PDX Furin inhibitor Signal Peptide (SEQ ID NO:2): FIG. 28E: SV40 promoter (SEQ ID NO:31); FIG. 28F: CMV immediate early promoter (SEQ ID NO:32); FIG. 28G: EF1 alpha promoter minimal (Ppef) (SEQ ID NO:33); FIG. 28H: EF1 alpha promoter genomic (SEQ ID NO:34); FIG. 28I: EF1 alpha promoter commercial (Oxford Genetics Ltd) (SEQ ID NO:35).

FIG. 29. Primers designed in order to delete a fragment of the MCS containing Lumio and 10× His tags (SEQ ID NO:36) (SEQ ID NO:37) (SEQ ID NO:38).

FIG. 30. Synthetic PuroR DNA sequence (SEQ ID NO:39) with internal HindIII and PmII restriction sites.

FIGS. 31A-31D. FIGS. 31A and 31B. DNA sequence of pPEF5PuroR (SEQ ID NO:40).

FIG. 31C. Plasmid map for pPEFPuroR; FIG. 31D: Feature map from pPEF5PuroR.

FIG. 32. Western Blot analysis of the complete conditioned media demonstrating THSD7A expression.

FIG. 33A Coomassie staining for protein integrity and purity for stable cell lines expressing Amyloid Precursor Protein (APP) a and B forms. FIG. 33B. analysis with isoform-specific neopepitope antibodies. FIG. 33C. comparison of the purified proteins with their commercial analogues.

FIG. 34. Coomassie staining of conditioned serum free medium showing high expression levels of Trastuzumab.

FIG. 35. Coomassie staining of conditioned serum free media revealing perceptible Trastuzumab expression in CHO-K1 cells.

FIG. 36. Coomassie staining revealing a higher expression level of Amyloid Precursor Protein beta form in CHO-K1 cells.

FIGS. 37A-37C. FIG. 37A. representation of EF1a constructs tested. FIG. 37B. DNA sequences of minimal EF1A1 proteins (SEQ ID NO:41); FIG. 37C. Primers (SEQ ID NO: 42) (SEQ ID NO:43) designed to delete regions from the 5′ end of the EF1a promoter in pPEF5.1 vector.

FIG. 38. Coomassie staining analysis of the conditioned media from all 3 stable cell line demonstrating a comparable expression level of PLA2R for pPEF5.11 and pPEF5.12 and no detectable expression for pPEF5.13 (SEQ ID NO:44) (SEQ ID NO:45) (SEQ ID NO:46) (SEQ ID NO:47) (SEQ ID NO:48).

### DETAILED DESCRIPTION

The present invention relates to in vitro expression of proteins and particularly, although not exclusively, to expression of proteins in mammalian cell lines. The present inventors have developed an expression vector which is useful for the expression of a wide range of proteins, including proteins that have historically proved challenging to express with adequate yield, or to express at all.

In one aspect, the present invention provides a nucleic acid expression vector that comprises a Gaussia signal peptide. The Gaussia signal peptide may be present as part of an expression cassette. The expression cassette may allow for insertion of nucleic acid encoding a gene of interest into the expression vector.

In some aspects, vectors utilise Gaussia signal peptide. Gaussia signal peptide has been used for a number of years to improve protein yield. However, the inventor has appreciated that inclusion of the signal peptide alone is not sufficient to ensure efficient recombination, but rather the context in which that signal peptide is present is also important. The inventor realised that other aspects of the vector must also be optimized in order to enhance the expression of the target protein, in addition to the presence of Gaussia signal peptide. The size of the vector and the stringency of selection are important aspects, particularly for vectors optimized for the expression of proteins and peptides that are normally considered to be challenging to produce using recombination expression. The inventor has determined that optimum protein yield may be obtained by using a vector comprising a Gaussia signal peptide and which is smaller than 5000 kb.

### Vectors

Vectors are autonomously replicating nucleic acid molecules that can be used to carry foreign nucleic acid fragments. Nucleic acid of interest is first cloned into an appropriate vector. The vector containing the nucleic acid of interest is then transfected into a host cell for expression. Expression of a nucleic acid of interest in mammalian cells usually utilizes vectors derived from mammalian viruses, or which include elements derived from human viruses, such as Simian Viruses 40 (SV40), polyomavirus, herpesvirus and papovirus. In order to construct vector the requirement is to select an efficient promoter and also the selection marker.

One type of vector is a plasmid. Plasmids are genetic structures that can replicate in a cell, independently of the chromosomes. Plasmids are typically small circular DNA strands in the cytoplasm. Particularly preferred plasmids herein are expression plasmids. Expression plasmids are plasmids designed for protein expression in cells. The plasmids are used to deliver nucleic acid encoding a protein of interest into cells, but are further able to interact with the cell to synthesize that protein, for example by utilizing the cell's protein synthesis machinery.

### Operably Linked

In this specification the term “operably linked” means that the promoter is in the correct location and orientation in relation to a nucleic acid that is to be transcribed to control the initiation of transcription by RNA polymerase.

In some cases, this may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. Where appropriate, the resulting transcript may then be translated into a desired protein or polypeptide.

Preferably, the nucleic acid expression vector of the present invention consists of fewer than 6000 base pairs of DNA. When the nucleic acid of interest, or gene of interest, is inserted into the vector, the size may be increased. However, preferably, the vector backbone (i.e. the elements of the vector which do not include the nucleic acid of interest or gene of interest) consists of fewer than 6000 base pairs. More preferably, the backbone consists of fewer than 5800 base pairs, more preferably fewer than 5600 base pairs, more preferably fewer than, more preferably fewer than 5400 base pairs, more preferably fewer than 5200 base pairs, more preferably fewer than 5000 base pairs, more preferably fewer than 4900 base pairs, more preferably fewer than 4800 base pairs, more preferably fewer than 4700 base pairs, more preferably fewer than 4600 base pairs, more preferably fewer than 4500 base pairs, more preferably fewer than 4400 base pairs, more preferably fewer than 4300 base pairs, more preferably fewer than 4200 base pairs, more preferably fewer than 4100 base pairs, more preferably fewer than 4000 base pairs, more preferably fewer than 3900 base pairs, more preferably fewer than 3800 base pairs, more preferably fewer than 3700 base pairs, more preferably fewer than 3600 base pairs, more preferably fewer than 3500 base pairs, more preferably fewer than 3400 base pairs, more preferably fewer than 3300 base pairs more preferably fewer than 3200 base pairs, more preferably fewer than 3100 base pairs, more preferably fewer than 3000 base pairs, more preferably fewer than 2900 base pairs, more preferably fewer than 2800 base pairs, more preferably fewer than 2700 base pairs, more preferably fewer than 2600 base pairs, more preferably fewer than 2500 base pairs. In some cases, the size of the backbone is minimised by removing elements that are not essential for transfection. For example, in some cases, the backbone does not contain a selectable marker, such as an antibiotic resistance gene.

In some cases, the vector backbone includes nucleic acid sequences encoding Ubiquitous Chromatin Opening Elements (UCOEs). UCOEs can give rise to populations of cells that express transgenes at highly reproducible and stable levels, to ensure sufficient expression of a protein of interest in one or more cell types. More than one UCOE sequence may be included in the vector. A UCOE sequence may be used to link two sequences of interest together, such as where antibody V_{H} and V_{L} sequences are expressed in the same vector. For example, the V_{H} sequence may be expressed under the control of a promoter, and the V_{L} sequence may be expressed under the control of a second promoter, with a UCOE separating the sequences.

### Expression Cassette

An expression cassette is part of a nucleic acid expression vector that directs a host cell's machinery to make RNA and protein from the vector. An expression cassette normally comprises nucleic acid encoding a promoter and a 3′ untranslated region, such as a polyadenylation sequence. The expression cassette preferably includes a multiple cloning site (MCS) for inserting nucleic acid of interest. The expression cassette may comprise the nucleic acid of interest. The expression cassette may include an open reading frame in which the nucleic acid of interest may be inserted. Alternatively, the expression cassette may be adapted for insertion of an open reading frame containing the nucleic acid of interest.

An open reading frame preferably includes an upstream start codon, and may include a downstream stop codon. The gene of interest, or the open reading frame, may be in frame with the promoter.

### Promoter

The vectors described herein use a promoter. A promoter is a region of nucleic acid that initiates transcription of a particular gene or downstream nucleic acid. Promoters may be located near the transcription start site of genes. In bacterial cells, promoters may be recognised by RNA polymerase in the cell, and an associated sigma factor, to initiate transcription. In Eukaryotic cells the process is more complicated, with at least seven different factors facilitating the binding of RNA polymerase II to the promoter. Promoters are critical elements of expression vectors, and may work in conjunction with other regulatory elements, such as enhancers, silencers and insulators to direct the level of transcription of a given gene. As such, different promoters may direct different levels of transcription.

In certain aspects of the present disclosure, the promoters that drive expression of the selectable marker are weak promoters. That is, they direct lower levels of transcription of a selectable marker gene than other promoters. In particularly preferred aspects, the promoter is weaker than the EF1A promoter. That is to say that the promoter directs lower levels of expression of the selectable marker than the EF1A promoter. The relative weakness of the promoter may be determined by methods known in the art, such as by methods disclosed in Qin et al., 2010 (PLOS ONE 5 (5): e10611; 1-4), the entire contents of which are incorporated herein by reference. The promoter may be weaker than EF1A when used in an identical vector context (i.e. all other elements of the vector are identical), and when expressed in the same cell type and under the same conditions. In this context, any suitable measure for promoter strength may be used, for example transcript number, or amount of protein produced.

Promoters useful for driving expression of the selectable marker in the nucleic acid vectors disclosed herein include SV40, UBC, PGK, TRE, CAGG or EF1A promoters. Particularly preferred promoters are SV40, UBC and PGK. In particularly preferred vectors, the promoter is SV40.

Nucleic acid vectors according to the invention also comprise a promoter to drive expression of the gene of interest. Preferably, this promoter is a strong promoter. In some cases, the promoter is a constitutive promoter. In other cases, the promoter is an inducible promoter, and only directs expression of the gene of interest in response to a particular stimulus.

Suitable constitutive promoters for driving expression of the gene of interest are known in the art, and include CMV.

Suitable inducible promoters are also known in the art, and are particularly useful in the expression of a gene of interest which is harmful to the cell in which it is to be expressed, such as a gene of interest encoding a toxic protein. By using intermittent expression of the toxic protein, negative effects on the growth and proliferation of host cells by the toxic protein may be minimised or avoided. Inducible promoters are those which initiate gene transcription in response to a stimulus, such as the presence of an inducing agent such as doxycycline (i.e. a doxycycline inducible promoter). An alternative strategy for expression of toxic proteins is to use riboswitches. A riboswitch is a regulator segment of a messenger RNA (mRNA) molecule that is able to bind a small molecule, resulting in a change in production of protein encoded by the mRNA. Thus, addition of the small molecule to the cell may stimulate production of the toxic protein from the mRNA, whereas the absence of the small molecule may allow the cells to grow and proliferate.

As used herein, “upstream” and “downstream” refer to a relative position in the nucleic acid sequence. Nucleic acid sequence has a 5′ end and a 3′ end. The 5′ end is the upstream end, and the 3′ is the downstream end. Transcription begins at the 5′ end and moves along the nucleic acid sequence towards the 3′ end. Where the nucleic acid is double stranded, such as with dsDNA, the terms are used in the context of the strand that encodes the gene of interest, or the “coding strand”.

The vector may comprise an untranslated region (UTR), such as a polyadenylation sequence (polyA tail), downstream of the MCS or nucleic acid of interest. This downstream sequence, or 3′ UTR is positioned immediately downstream of the stop codon of the open reading frame. The 3′ UTR may comprise regulatory regions that influence polyadenylaton, translation efficiency, localisation and stability of mRNA translated from the expression vector. The 3′ UTR may encode a polyA tail sequence, which may be important for nuclear export, translation and stability of mRNA transcribed from the vector. The 5′ UTR may contain a translational enhancer sequence. The translational enhancer sequence may be the SP163 translational enhancer sequence.

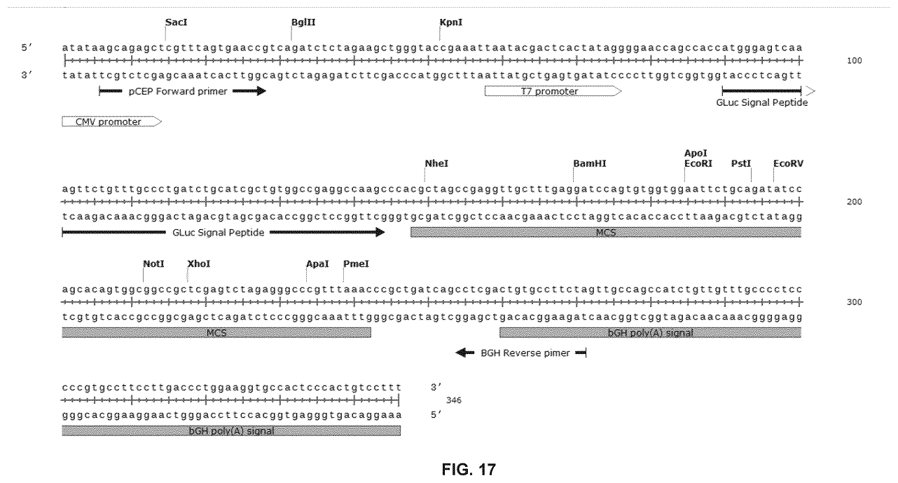

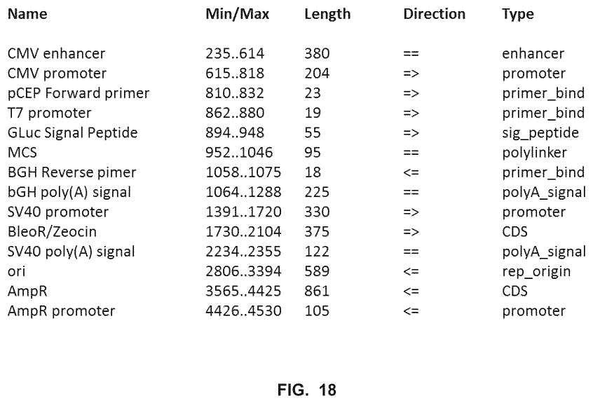

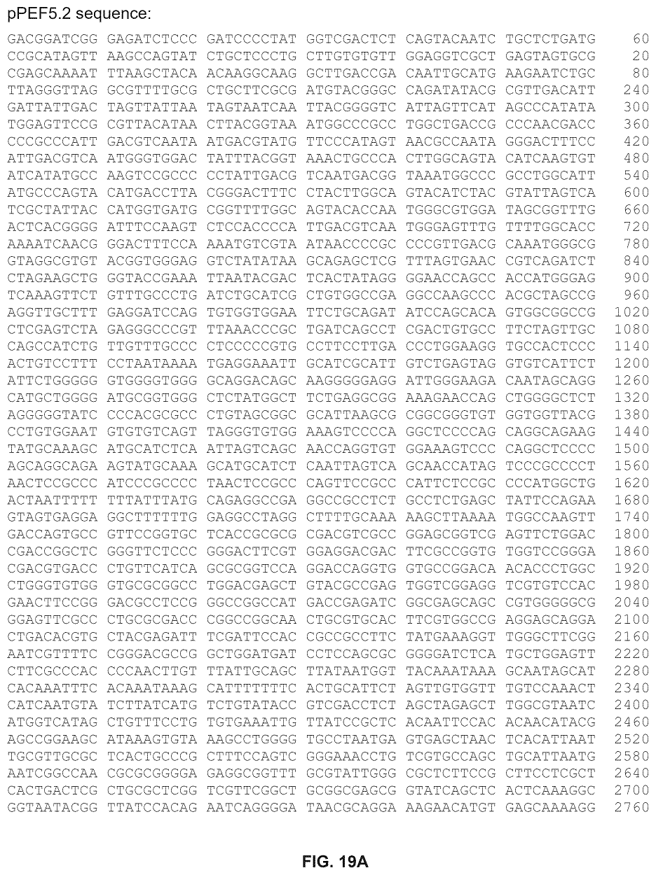

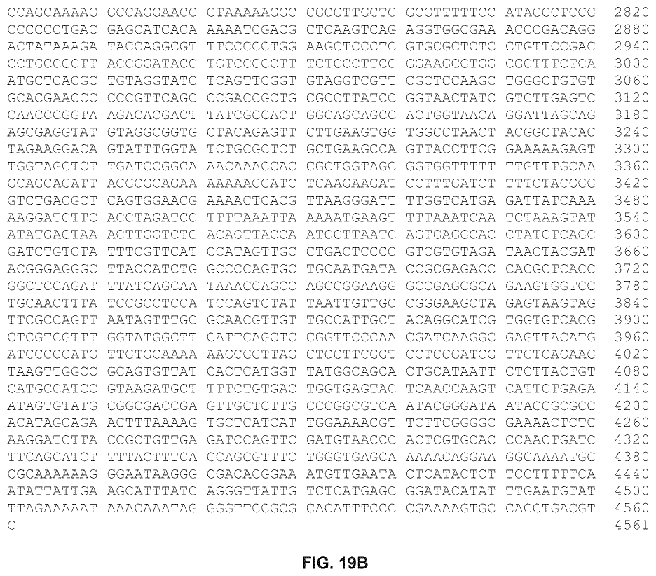

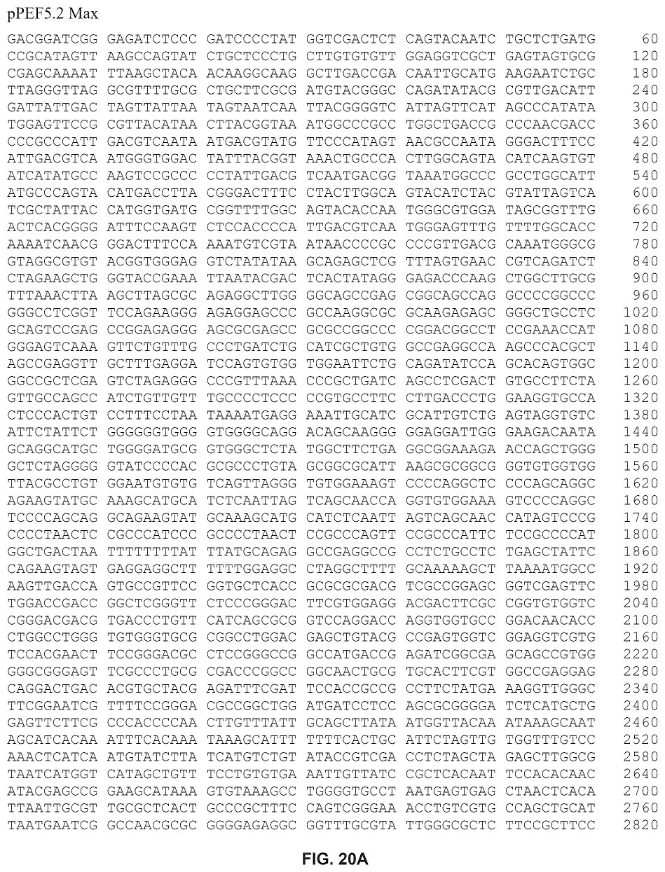

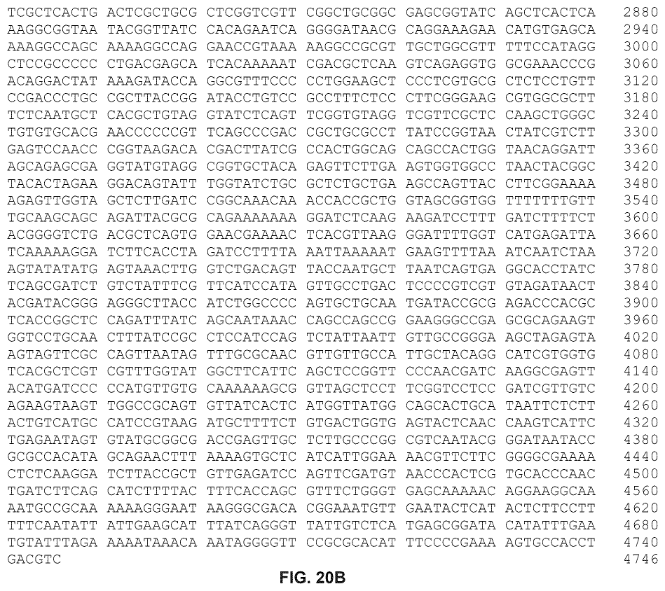

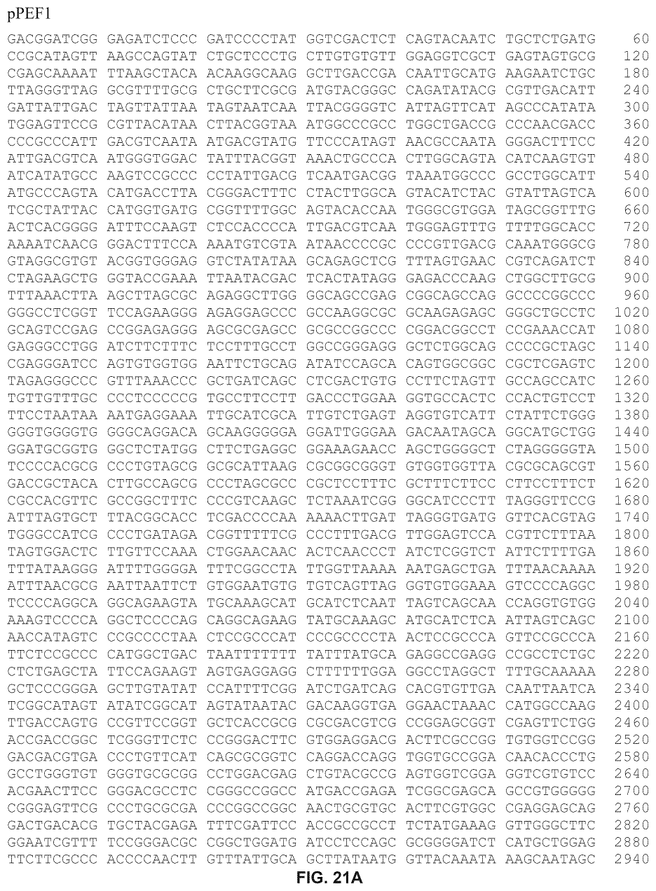

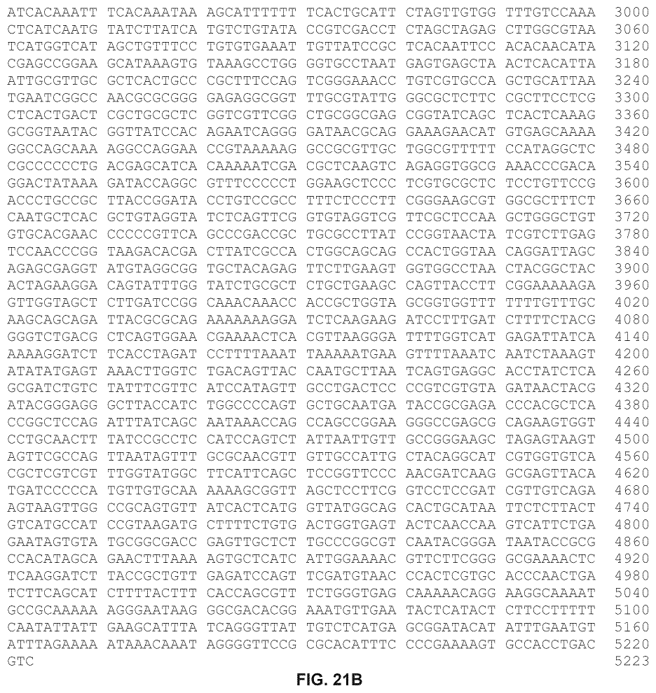

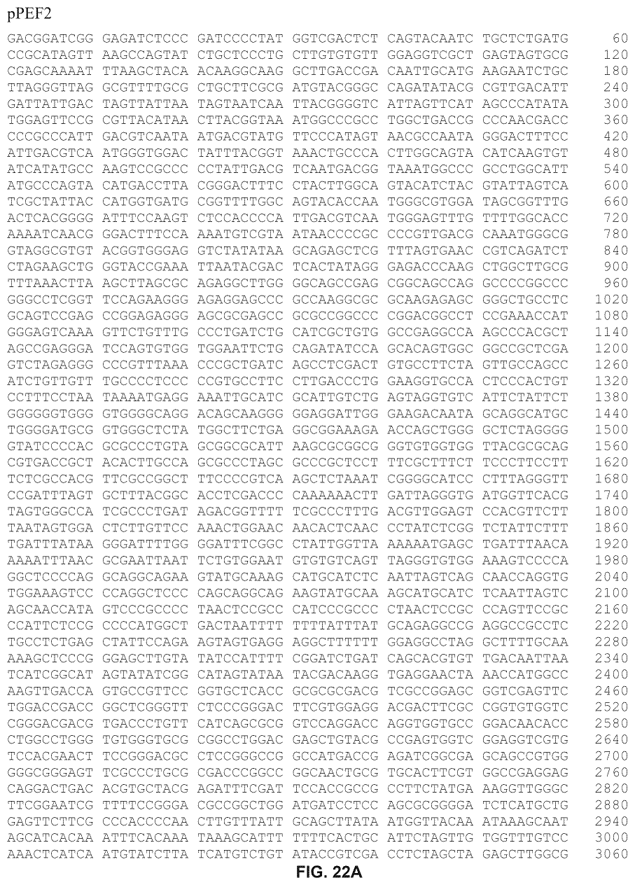

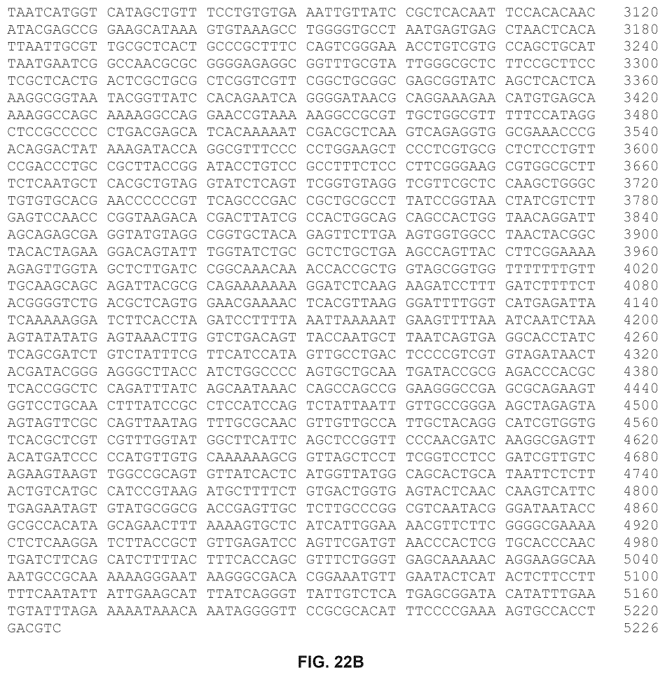

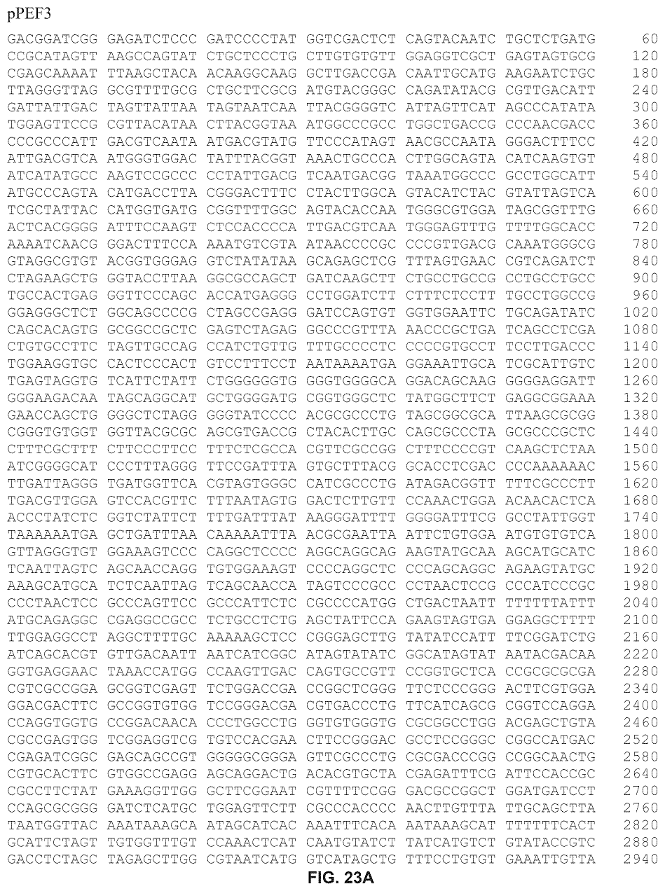

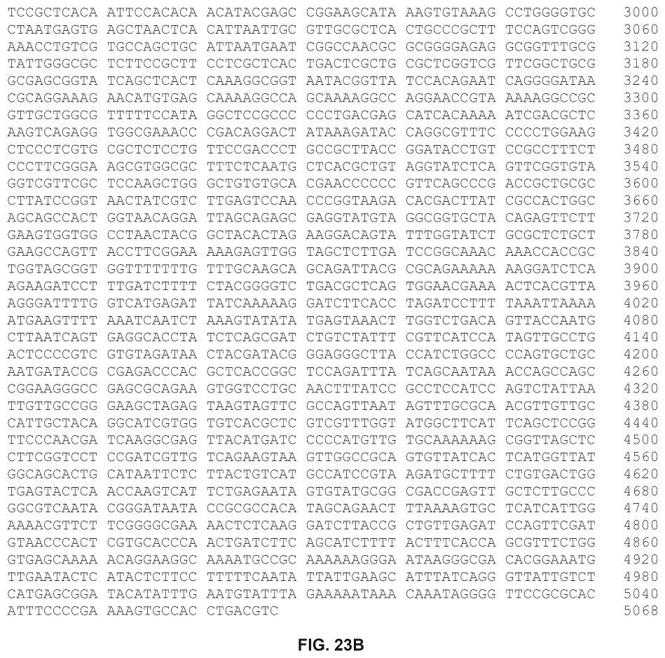

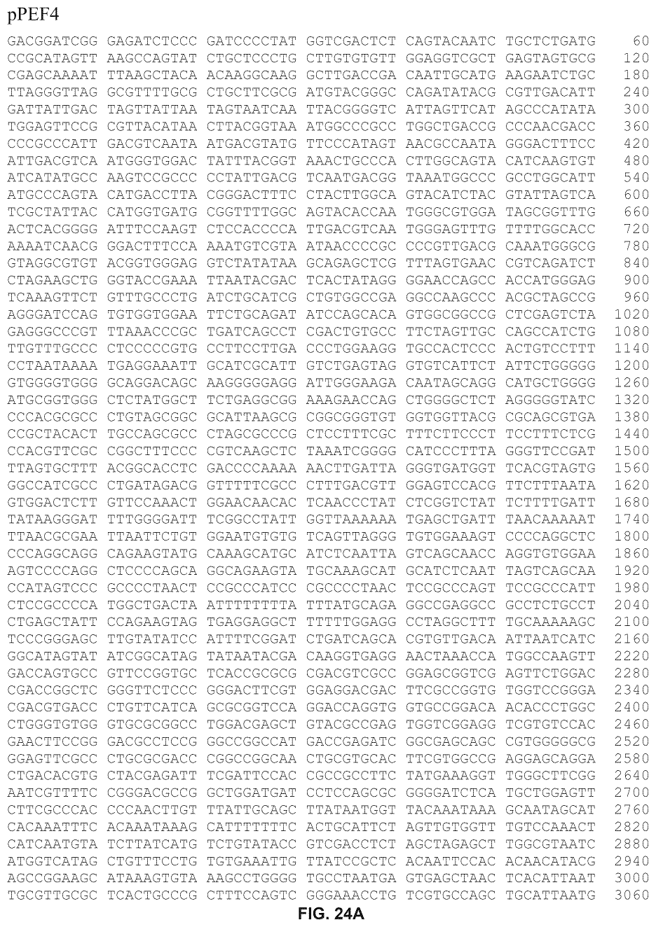

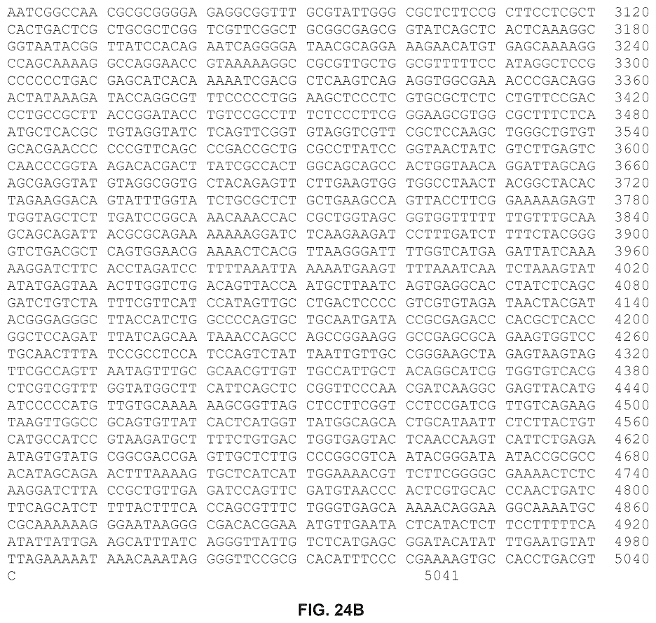

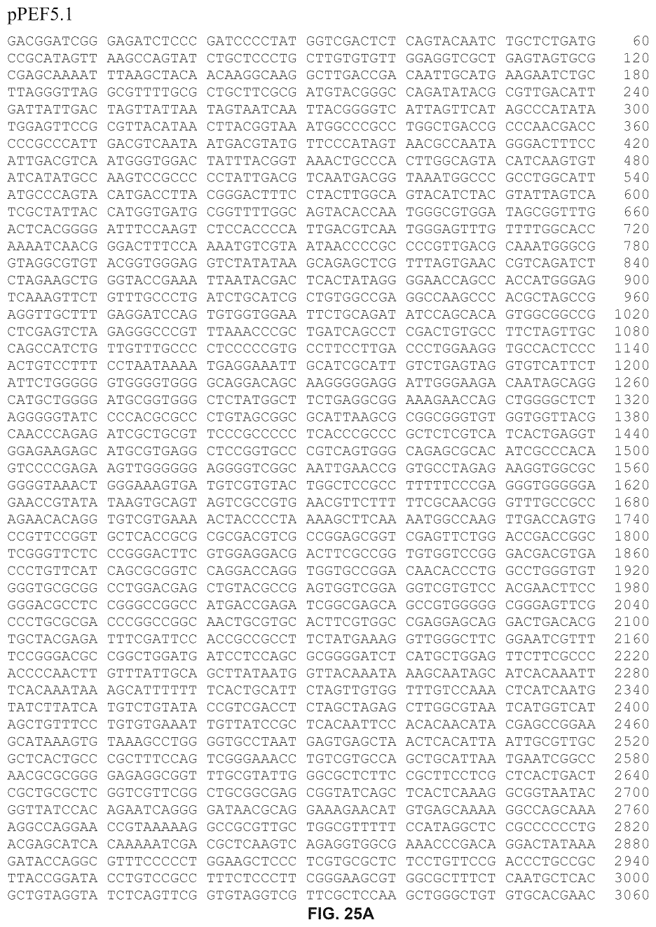

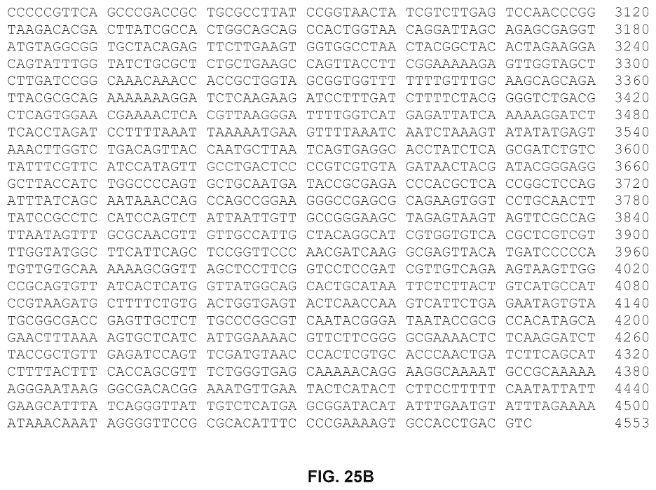

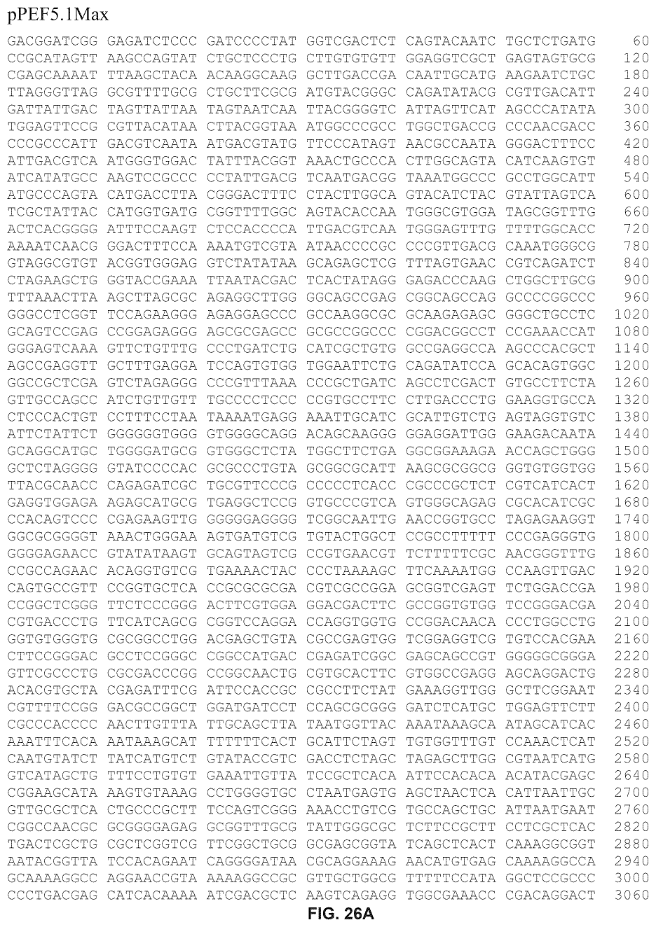

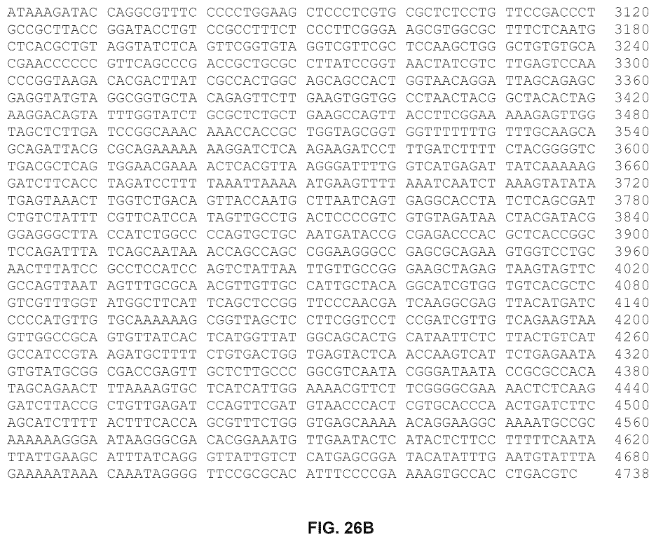

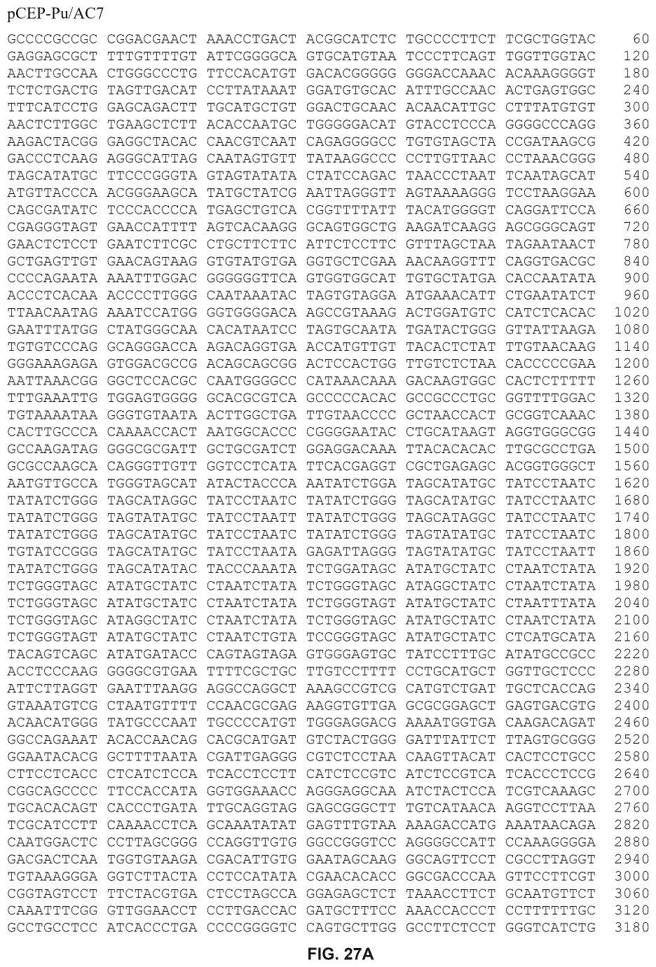

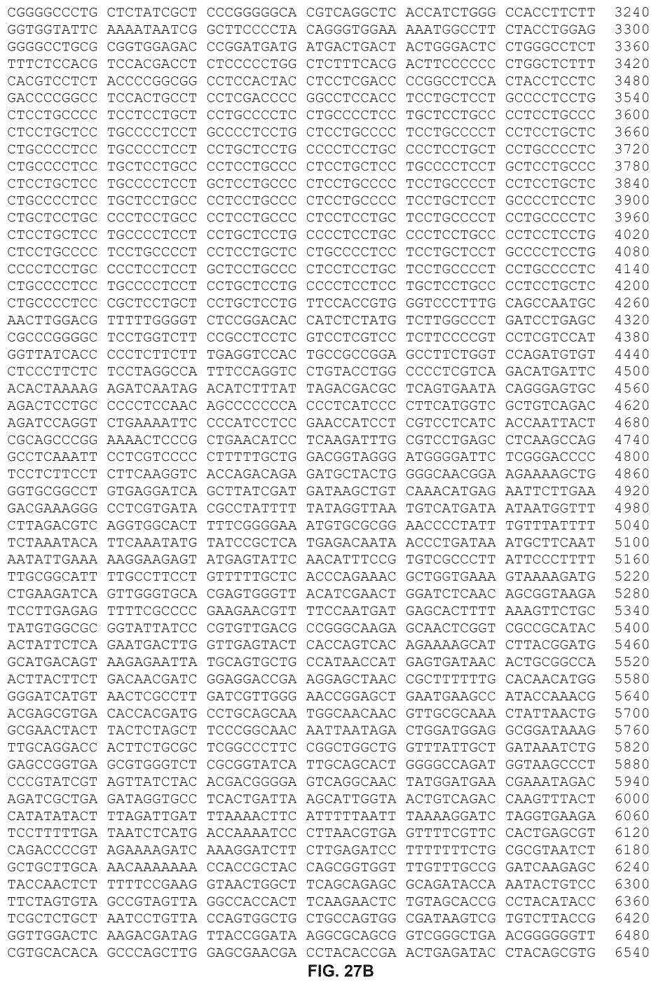

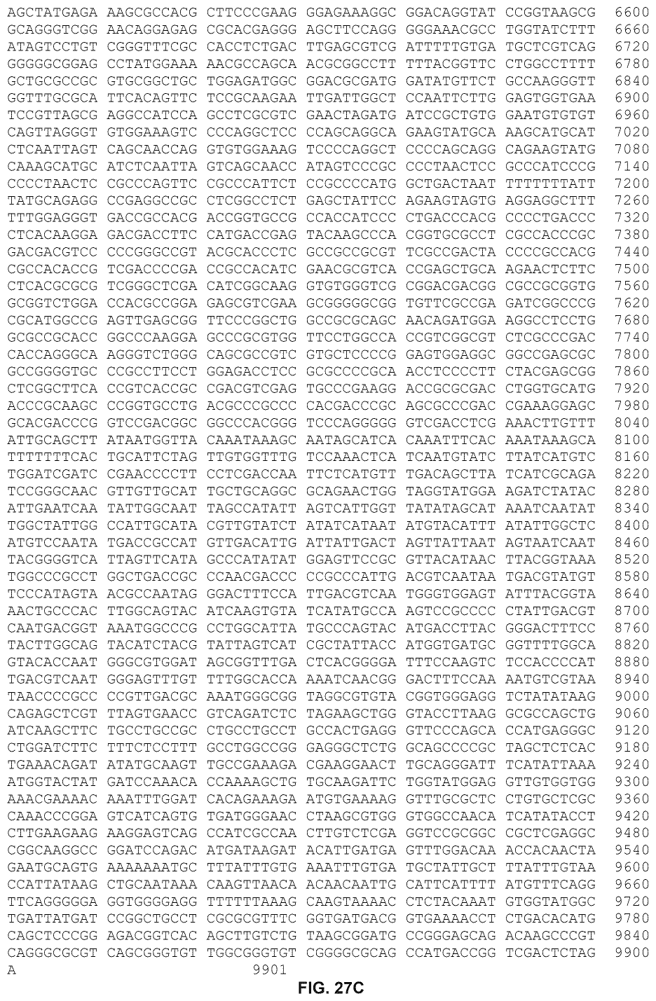

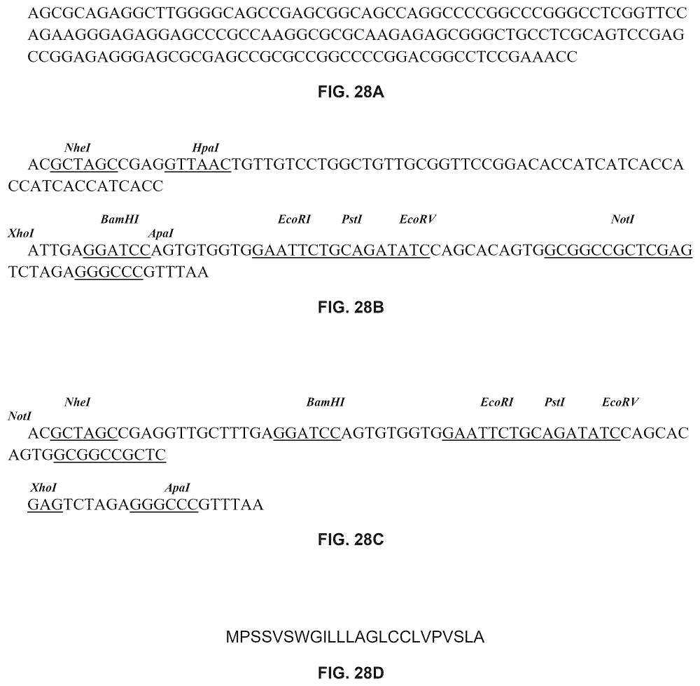

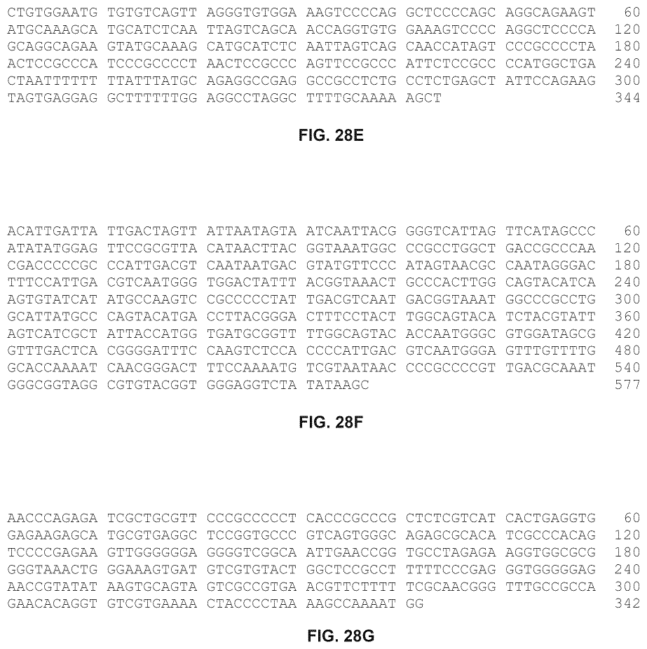

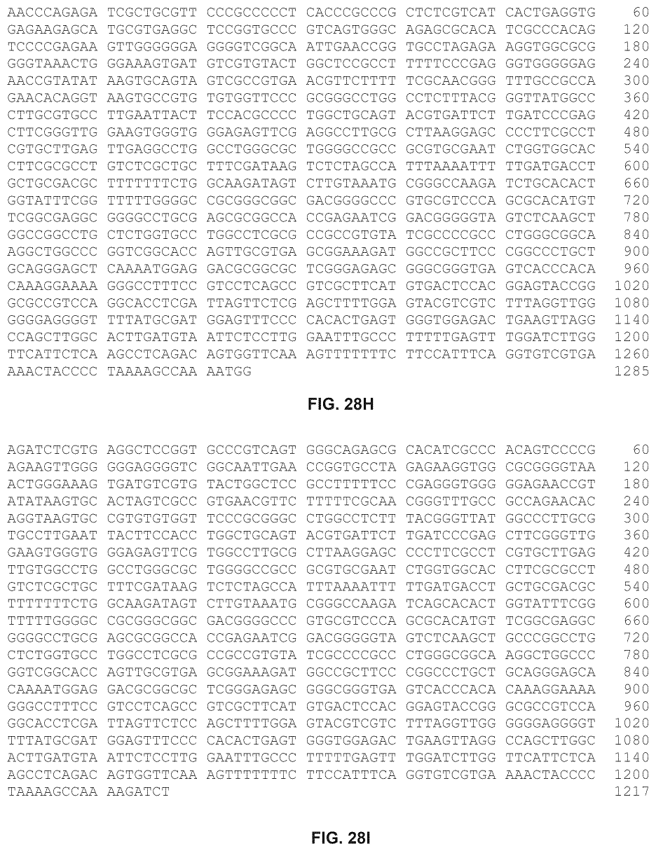

The vector may comprise a multiple cloning site (MCS). A multiple cloning site may also be referred to as a polylinker. The MCS contains a plurality of restriction sites. The multiple cloning site may be as shown in FIG. 17. It may comprise the sequence set out in SEQ ID NO: 16.

In one aspect, the invention provides a pPEF vector comprising a Gaussia signal peptide.

Nucleic acid expression vectors according to the present invention comprise nucleic acid encoding a signal peptide. A signal peptide is a short nucleic acid sequence present in the N-terminus of newly synthesised proteins. The signal peptide may be present in the N-terminus of proteins that are to be secreted. The signal peptide may direct the protein to the plasma membrane.

Preferably, the nucleic acid expression vectors of the present invention comprise nucleic acid encoding the signal peptide of Gaussia luciferase, referred to herein as Gaussia signal peptide, or GLUC. Gaussia luciferase signal peptide may have a sequence MGVKVLFALICIAVAEA (SEQ ID NO:1).

In another aspect, the vectors comprise nucleic acid encoding the signal peptide of PDX-Furin inhibitor. The signal peptide of PDX-Furin inhibitor may have the sequence MPSSVSWGILLLAGLCCLVPVSLA (SEQ ID NO:2).

### Selectable Marker

In some aspects, nucleic acid vectors disclosed herein comprise nucleic acid encoding a selectable marker. A selectable marker is a gene introduced into a cell that confers a trait suitable for artificial selection. Selectable markers include antibiotic resistance genes. In some aspects, the nucleic acid vectors disclosed herein include nucleic acid encoding genes that infer resistance to puromycin, ampicillin, Zeocin® antibiotic, kanamycin, blasticidin, geneticin (G418), neomcyin, hygromycin, chloramphenicol or tetracycline.

### Tags

In some aspects, the vector also includes nucleic acid encoding a tag. Tags may be useful for isolating the protein of interest, or may facilitate expression of the protein of interest. Suitable tags include GST (Glutathione-S-Transferase)-tag, HA-tag, Myc-tag, His-tag, V5-tag, Flag-tag, CBP (chitin binding protein)-tag, MBP (maltose binding protein)-tag, GFP-tag and its modifications, RFP-tag and its modifications, YFP-tag and its modifications, Lumio-tag, Nus-tag, Streptavidin-tag, T7-tag, S-tag, thioredoxin-tag and Softag. In certain aspects, the vector comprises a SUMO tag.

### SUMO

SUMOstar is a technique developed by LifeSensors (www.lifesensors.com). SUMO-tag expression systems can maximize the yield of soluble, functional proteins in E. coli, yeast, insect and mammalian cells. SUMO functions as both a chaperonin and as an initiator of protein folding to dramatically improve the solubility and level of expression of your protein of interest. SUMO may be particularly useful in the expression of small proteins. For example, expression of nucleic acid encoding Noggin. SUMO may be incorporated into the vector.

Alternatively, SUMO may be fused in frame to the nucleic acid of interest which is inserted into the vector.

### Cells

The nucleic acid vectors disclosed herein are useful for the expression of peptides in cells. Any cell suitable for the expression of polypeptides may be used for producing peptides according to the invention. The cell may be a prokaryote or eukaryote. Preferably the cell is a eukaryotic cell such as a yeast cell, a plant cell, insect cell or, most preferably, a mammalian cell. In some cases the cell is not a prokaryotic cell because some prokaryotic cells do not allow for the same post-translational modifications as eukaryotes. In addition, very high expression levels are possible in eukaryotes and proteins can be easier to purify from eukaryotes using appropriate tags. Specific plasmids may also be utilised which enhance secretion of the protein into the media.

Preferably the methods and plasmids of the invention relate to expression in mammalian cells. Any mammalian cell may be used. Suitable mammalian cells include established cell lines, including Human embryonic kidney cells (HEK293, particularly HEK293T cells, but also HEK293-EBNA1 cells and HEK293/TR cells), Chinese Hamster Ovary (CHO), CHO cell derivatives such as CHO-K1, CHOpro-3, and FreeStyle CHO-S cells, DUKX-X11, DG44, COS cells (monkey kidney cells), including COS-1 and COS-7 cells, Vero cells (African monkey kidney cells), Hela cells (cervical adenocarcinoma cells), NSO cells (murine melanoma), Jurkat cells (immortalized human T lymphocytes), BHK cells (baby hamster kidney) and MCF cells (Michigan Cancer Foundation) such as MCF-7 cells, PER.C6, and L cell mouse fibroblasts such as those deposited at ATCC CRL 2648. In some cases, the cells are a human cell line.

### Protein Expression

The methods and vectors of the present invention are suitable for the expression of any protein. They are particularly suited to proteins which have proved difficult to express in conventional protein expression vectors and systems. Nucleic acid encoding the protein of interest (referred to herein as the nucleic acid of interest, nucleic acid encoding the gene of interest) is introduced into the vector, and the vector is transfected into a cell. The protein is then produced by the cell.

Proteins particularly suited to the methods and plasmids of the present invention include antibodies, cytokines and growth factors, large proteins, secreted proteins, membrane proteins, kinases and receptors, and metalloproteinases.

The plasmids of the present invention are particularly useful for the expression of antibodies in mammalian cells.

Large proteins which may be expressed using the plasmids of the invention include proteins larger than THSD7A (Thrombospondin Type 1 Domain containing 7A; GenBank: NM_015204.2 GI: 259013332) and PLA2R (Pongo abelii phospholipase A2 receptor 1; GenBank: NM_001132708.1 GI: 197098169). The protein may be at least 1000 amino acids, at least 1200, at least 1400, at least 1600, at least 1800, at least 2000, or at least 2200 amino acids in length.

In other aspects, the protein is expressed as a fusion protein with a tag or other peptide sequence. In such cases, the fusion protein may be at least 1000 amino acids, at least 1200, at least 1400, at least 1600, at least 1800, at least 2000, or at least 2200 amino acids in length. Expression as a fusion protein may be particularly suitable where the protein to be expressed is a small protein or peptide. For example, the peptide may be 10 to 500 amino acids, 50 to 500, 100 to 400, 100 to 300, or 150 to 250 amino acids in length. The small protein may be expressed as a fusion protein with a tag which increases the size of the protein to be expressed. The tag may be removable from the expressed protein. Suitable tags include SUMO or Fc tags.

Secreted proteins which may be expressed using the plasmids of the invention include sVAP1 (synaptosomal associated protein; GenBank: NM_053052.3 GI: 224465201) and CD73 (GenBank: BC065937.1 GI: 42406318), and BMP4 (GenBank P12644; GI 115073). Cytokines and growth factors that may be expressed using the plasmids of the invention include Noggin (GenBank: U31202.1 GI: 1117816) and IFNb-1a (Interferon beta 1a; DrugBank: DB00060 (BIOD00093, BTD00093). Metalloproteinases that may be expressed using the plasmids of the invention include MMP12 (matrix metallopeptidase 12; GenBank: NM_002426.4 GI: 261878521). Kinases and receptors that may be expressed using the plasmids of the invention include RET (GenBank: KR709953.1 GI: 823670247). An example of a membrane protein is Heparanase (GenBank Q9Y251.2 GI 296434532).

In some cases, the vector may be used to express a toxic protein. For expression of toxic proteins, it is advisable to use an inducible promoter. Use of an inducible promoter means that expression of the protein may be intermittent, rather than constitutive. By using intermittent expression of the toxic protein, negative effects on the growth and proliferation of host cells by the toxic protein may be minimised or avoided. Inducible promoters are those which initiate gene transcription in response to a stimulus, such as the presence of an inducing agent such as doxycycline.

An alternative strategy for expression of toxic proteins is to use riboswitches. A riboswitch is a regulator segment of a messenger RNA (mRNA) molecule that is able to bind a small molecule, resulting in a change in production of protein encoded by the mRNA. Thus, addition of the small molecule to the cell may stimulate production of the toxic protein from the mRNA, whereas the absence of the small molecule may allow the cells to grow and proliferate.

Methods of producing a peptide of interest may involve culture or fermentation of a cell modified to express the peptide. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted proteins. Culture, fermentation and separation techniques are well known to those of skill in the art.

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culture of cells that express peptide of interest, that peptide is preferably isolated. Any suitable method for separating proteins from cell culture known in the art may be used. In order to isolate a protein of interest from a culture, it may be necessary to first separate the cultured cells from media containing the protein of interest. If the protein of interest is secreted from the cells, the cells may be separated from the culture media that contains the secreted protein by centrifugation. If the protein of interest collects within the cell, for example in the vacuole of the cell, it will be necessary to disrupt the cells prior to centrifugation, for example using sonication, rapid freeze-thaw or osmotic lysis. Centrifugation will produce a pellet containing the cultured cells, or cell debris of the cultured cells, and a supernatant containing culture medium, cell lysate and the protein of interest.

It may then be desirable to isolate the protein of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography, affinity chromatography, gel filtration, hydrophobic interactions, and reverse phase chromatography. These may be used as an alternative to precipitation, or may be performed subsequently to precipitation.

Once the protein of interest has been isolated from culture it may be necessary to concentrate the protein. A number of methods for concentrating a protein of interest are known in the art, such as ultrafiltration, lyophilisation or centrifugal concentration.

### Sequence Identity

In certain aspects the invention concerns compounds which are nucleic acids comprising a sequence having a sequence identity of at least 70% with a given sequence. Alternatively, this identity may be any of 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity.

Percentage (%) sequence identity is defined as the percentage of nucleic acid residues in a candidate sequence that are identical with residues in the given listed sequence (referred to by the SEQ ID No.) after aligning the sequences and introducing gaps if necessary, to achieve the maximum sequence identity, and not considering any conservative substitutions as part of the sequence identity. Sequence identity is preferably calculated over the entire length of the respective sequences.

Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence.

For example, where a given sequence comprises 100 nucleic acids and the candidate sequence comprises 10 nucleic acids, the candidate sequence can only have a maximum identity of 10% to the entire length of the given sequence. This is further illustrated in the following example:

### (A)

Given seq:
XXXXXXXXXXXXXXX
(15 nucleic acids)
Comparison seq:
XXXXXYYYYYYY
(12 nucleic acids)

The given sequence may, for example, be that encoding the plasmid as a whole, or an element within the plasmid, such as a sequence encoding a promoter.

% sequence identity=the number of identically matching nucleic acid residues after alignment divided by the total number of nucleic acid residues in the longer given sequence, i.e. (5 divided by 15)×100=33.3%

Where the comparison sequence is longer than the given sequence, sequence identity may be determined over the entire length of the given sequence. For example:

(B) Given seq:
XXXXXXXXXX
(10 nucleic acids)
Comparison seq:
XXXXXYYYYYYZZYZZZZZZ
(20 nucleic acids)

Again, the given sequence may, for example, be that encoding the plasmid as a whole, or an element within the plasmid, such as a sequence encoding a promoter.

% sequence identity=number of identical nucleic acids after alignment divided by total number of nucleic acids in the given sequence, i.e. (5 divided by 10)×100=50%.

Alignment for purposes of determining percent nucleic acid sequence identity can be achieved in various ways known to a person of skill in the art, for instance, using publicly available computer software such as ClustalW 1.82. T-coffee or Megalign (DNASTAR) software. When using such software, the default parameters, e.g. for gap penalty and extension penalty, are preferably used.

Identity of amino acid sequences may be determined in a similar manner involving aligning the sequences and introducing gaps if necessary, to achieve the maximum sequence identity, and calculating sequence identity over the entire length of the respective sequences. Where the aligned sequences are of different length, sequence identity may be determined as described above and illustrated in examples (A) and (B).

### Kits

Also disclosed herein are kits. Kits may comprise a nucleic acid expression vector as disclosed herein and one or more reagents. Kits may include reagents suitable for cloning a nucleic acid of interest into the vector, and/or reagents for transfecting a host cell with the vector. The kit may include a host cell. Kits may also comprise instructions for use.

### Methods

Disclosed herein are methods of making and using the nucleic acid expression vectors disclosed herein.

In one aspect, the disclosure provides a method of making an expression vector, the method comprising modifying a pPEF series vector to include a Gaussia signal peptide. The methods may involve reducing the size of the vector. The method may include removing non-essential sequences of the vector. The method may involve removing the f1 origin of replication. The method may involve removing the EM7 promoter. The method may involve introducing a transcriptional enhancer, such as an SP163 transcriptional enhancer. The method may also involve removing elements that are not essential for transfection, such as the selectable marker. For example, the method may involve the removal of an antibiotic resistance gene.

The method may involve removing the selectable marker and the promoter that controls the expression of the selectable marker.

In another aspect, methods of transforming a cell are provided. The methods may involve introducing a nucleic acid expression vector as disclosed herein. The methods may involve introducing the expression vector into a host cell, and then subjecting the host cell to selection, such as antibiotic selection.

In some aspects, methods involve the production of a protein of interest. Such methods involve transforming a cell with a nucleic acid expression vector disclosed herein that contains nucleic acid encoding the protein of interest. The method may involve selecting transformed cells, such as by antibiotic selection or nucleic acid analysis. In some case, the method involves inducing transient expression of the nucleic acid of interest. The method may involve separating the protein of interest from the cells.

The method may involve lysing the cells to extract the protein of interest. In preferred aspects, the protein of interest is secreted from the cells. Such methods may involve isolating the protein of interest from the medium in which cells are cultured. Methods of isolating and extracting proteins are known in the art. Any suitable method for protein isolation or extraction may be used with the vectors and host cells disclosed herein, and may depend on the protein to be isolated or extracted.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### EXAMPLES

### Example 1: Vector Optimisation

Our Protein Expression Facility (PEF) was established to produce milligrams of various proteins, which usually would not be available commercially, with the least costs for the researchers using our services. Back in 2010, when the mammalian expression platform was introduced in the Facility in addition to microbial and insect ones, it was chosen to use HEK293 EBNA1:pCEP stable episomal expression as one of the best described in the literature. It was shown to outperform stable expression based on pcDNA™3.1 vector when used with HEK293 cells and its derivatives (Durocher et al, 2002). However, in practice this system was not delivering consistently sufficient amounts of protein when used for different types and subclasses. Additionally, the newly created cell lines could lose their expression after a single freeze-thaw event. Since the employment of commercially available suspension grown strains and specially formulated media, although guaranteed to increase the recombinant yield, was not affordable, it was decided to focus our R&D on vector optimisation.

Vector design can significantly affect transcription and processing of the transcript, export of mRNA from the nucleus, targeting of mRNA, its stability and translation. This can be achieved through manipulations with promoters, 5′ and 3′ end UTRs and various DNA elements which enhance transcription such as Matrix Attachment Regions (MARs) and Ubiquitous Chromatin Opening Elements (UCOEs) to name some. One of the feasible to us approaches would be to concentrate our efforts on signal peptide optimisation as one of the most promising tools to increase recombinant yield. Literature search at the time highlighted the signal peptide of a marine copepod, Gaussia princeps, as a very promising one. The original work of Tannous and co-workers in 2005 showed that Gaussia luciferase (GLuc) is very efficiently secreted in mammalian cells. Several reports published later demonstrated that GLuc signal peptide consistently and significantly, up to 100 fold, outperformed various mammalian secretory signal peptides as well as the ones from other marine organisms (Knappskog et al, 2007; Stern et al, 2007; Wen et al, 2011). When compared to its mammalian counterparts, GLuc was shown to impact on protein production at post-transcriptional level, possibly through enhancement of mRNA-protein (mRNP) formation and transport to translocons. Tested together with the well-known mammalian signal peptide routinely used in the Facility, BM40 (Swaroop et al., 1988), it was thought to give us a good idea whether we are already utilizing the best approach for the production of soluble recombinant proteins or there is still some scope for an increase which we could benefit from when dealing with difficult-to-express targets.

Inspiration for introducing another variable, a vector different to pCEP-Pu, came from our previous experiments. In 2010, when trying to increase the production of a soluble Phospholipase A2 Receptor (PLA2R1) presented in the Examples section, we attempted the generation of double stable lines. The original episomal pCEP-Pu:PLA2R1 line yielded very low amounts of protein, approximately 100 microgram per litre on average. Since even this low yield would be lost upon freezing, new stable lines had to be created regularly to meet the demand. In addition to the full-length soluble PLA2R1, there were truncated forms which together exceeded the amount of the full-length protein. We speculated that Furin would be one of the reasons for the truncation. It is a common proprotein convertase which cleaves proteins destined for secretion. By blocking Furin we hoped to see the increase in the yield of the full-length PLA2R1 at the expense of diminishing truncated forms. We procured a construct containing alpha-1 PDX (Jean et al., 1998), a furin inhibitor, which came from Prof. Kidd's lab in the USA (Columbia University).

The a1-PDX coding sequence was subcloned into the following mammalian expression vectors:
a) pCEP-Pu (pCEP-Pu/AC7 (Kohfeldt et al, 2007), based on pCEP4 (Invitrogen)—the most popular vector for episomal expression);
b) pcDNA™4/HisMax (Invitrogen) vector, an advanced version of pcDNA™3.1, which is a popular vector for stable expression;
c) pRTS-1 (Bornkamm et al., 2005), a kind gift of Prof. Dirk Eick, German Research Centre for Environmental Health (Munich).

pCEP-Pu vector was chosen for transient transfection of the pCEP-Pu:PLA2R1 stable cell line. pcDNA™4/HisMax vector contained a Zeocin® antibiotic resistance gene, whereas pRTS-1 had a hygromycin resistance gene. Both allowed creation of double stable cell lines since the basal pCEP-Pu:PLA2R1 line had been selected via puromycin resistance.

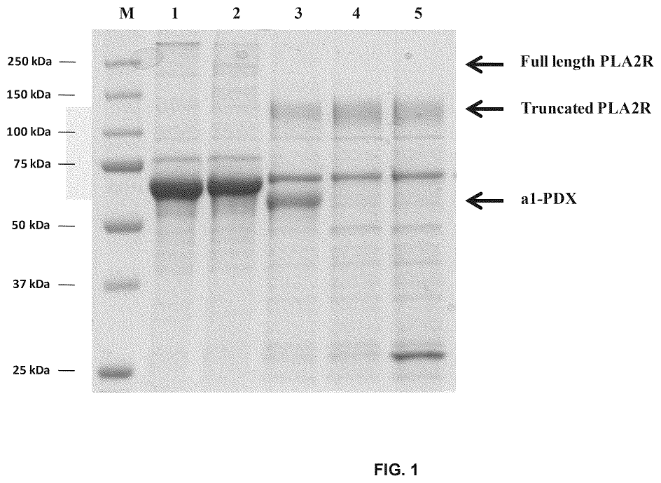

FIG. 1 shows the results of a1-PDX expression in double stable lines. While the effort did not increase the yield of the full-length PLA2R1 but, on the contrary, enhanced the production of the truncated PLA2R1 form, it did highlight pcDNA™4/HisMax vector as the most efficient for the expression of a1-PDX protein.

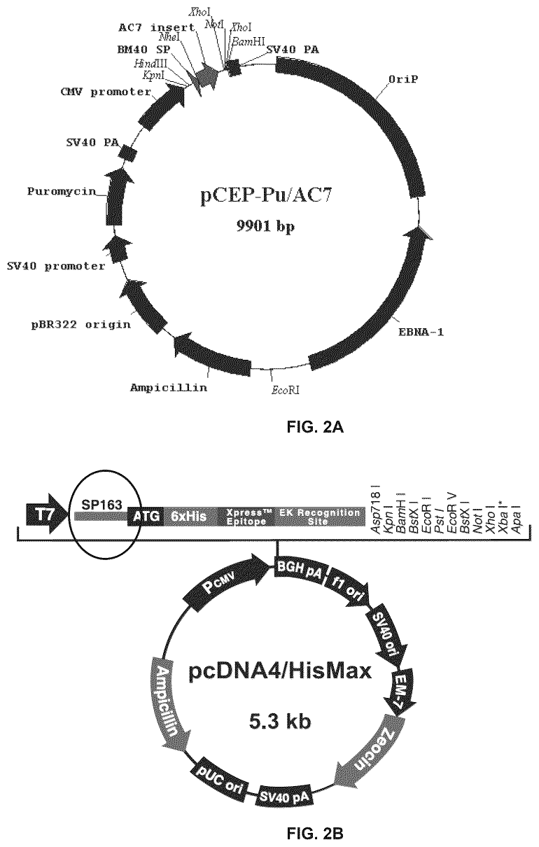

Thus, it was decided to concentrate our further R&D effort on two vectors, pCEP-Pu:AC7 and pcDNA™4/HisMax, two Signal Peptides (SP), BM40 (MRAWIFFLLCLAGRALA) (SEQ ID NO: 3) and Gluc (MGVKVLFALICIAVAEA) (SEQ ID NO:1) and their combination with the SP163 Transcriptional enhancer which present in pcDNA™4/HisMax vector as shown in FIG. 2B.

We chose a secreted form of Vascular Adhesion Protein-1 (sVAP-1), as the model protein for our expression studies which is described in the Examples section. It has been the best protein in our portfolio in terms of expression stability and high yield producing up to 60 mg/L (Heuts et al., 2011). The cloning strategy was as follows:

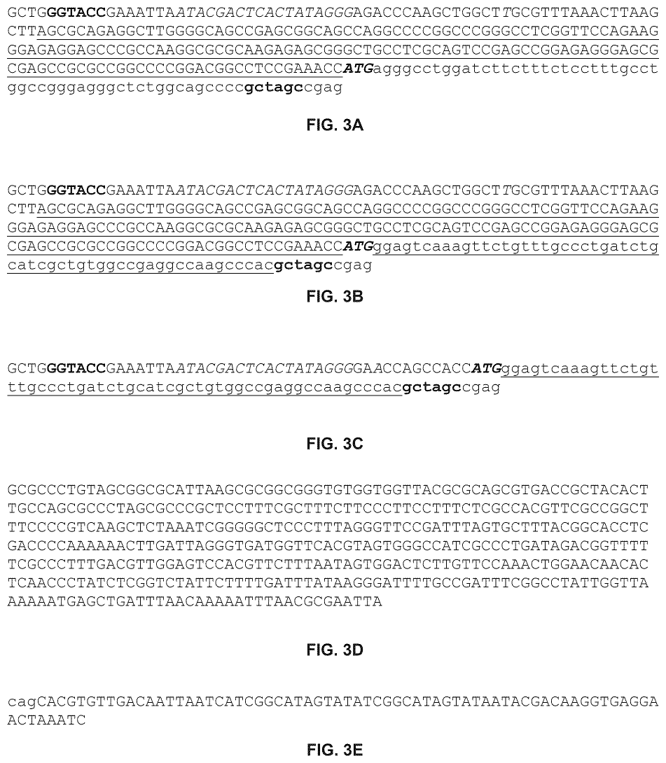

We used the existing pCEP-Pu:sVAP-1 vector as the basis (described in the cited above paper and in the Examples section). The construct was digested with KpnI and NheI to drop the BM40 signal peptide and one of the 3 synthetic fragments presented in FIGS. 3A-3C was inserted.

Then we used the original pCEP-Pu:sVAP-1 (with the BM40 signal peptide) and the newly created pCEP-sVAP-1 constructs with fragments 1, 2 and 3 to excise a part of the Pcmv promoter and the ligated fragments with sVAP-1 after NdeI/BamHI digestion. Those larger fragments were ligated into NdeI/BamHI digested pcDNA™4/HisMax C vector.

The resulting pcDNA™4 based constructs should be named as follows: pPEF1 for pcDNA™4-SP163-BM40; pPEF2 for pcDNA™4-SP163-GLuc; pPEF3 for pcDNA™4-BM40 and pPEF4 for pcDNA™4-GLuc.

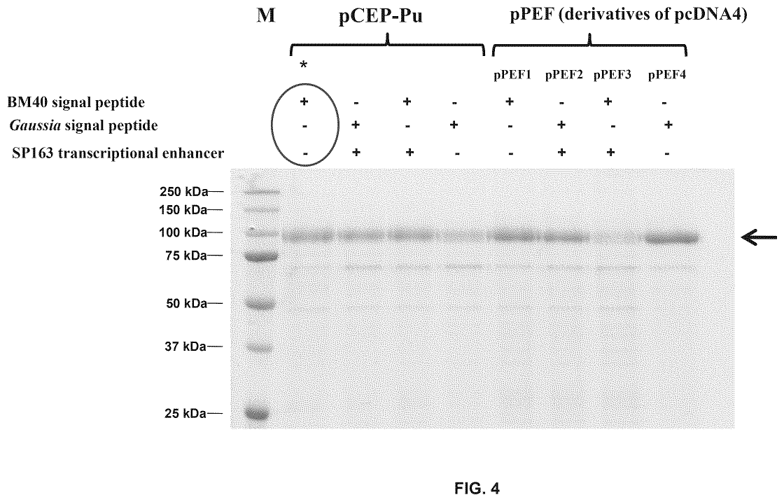

The expression constructs, pCEP-Pu based and pcDNA™4 derived, were transfected into HEK293 EBNA1 cells with Lipofectamine™ transfection reagent (Invitrogen/Life Technologies) following the manufacturer's instructions. A selective reagent (puromycin for pCEP-Pu derived constructs and Zeocin® antibiotic for pPEF1-4) was added 48 hours post-transfection. Pools of stable colonies were established and expanded after 3-4 weeks of culturing in case of pPEF vectors and 4-5 weeks for pCEP expression plasmids. Once the stable cells reached 100% confluency in a T25 culture flask, the complete medium was replaced with serum-free one (SFM) and cultured for 7 days. The SFM was then harvested and analysed for the presence of a secreted protein either by Coomassie staining or Western blotting (anti-His). FIG. 4 demonstrates the differential expression of sVAP-1 in various constructs with the pPEF suite showing an increase compared to the pCEP vectors.

Since the pPEF vectors, especially the ones incorporating GLuc, revealed a promising trend for enhancing protein production, it was chosen to test it further with PLA2R1 as the most difficult target in terms of low yield and reproduction (see the Examples section for the original cloning of pCEP-Pu:PLA2R1 and purification details). To generate pPEF1-4:PLA2R1, the corresponding constructs of pPEF:sVAP-1 were digested with NheI/NotI to drop the sVAP-1 coding sequence and replace it with the PLA2R1 excised with the NheI/NotI from the original pCEP-Pu:PLA2R1 (Kanigicherla et al., 2013). To create the pCEP-Pu suite of constructs, additional cloning steps had to be introduced in view of very limiting MCS of this vector backbone. The pCEP-Pu:AC7 vector was digested with KpnI and NheI to incorporate the synthetic fragments given in FIGS. 3A-3C, also KpnI/NheI treated. The resulting pCEP-Pu derived vectors were NheI/NotI digested to omit the AC7 insert and to be ligated with PLA2R1 coding sequence instead.

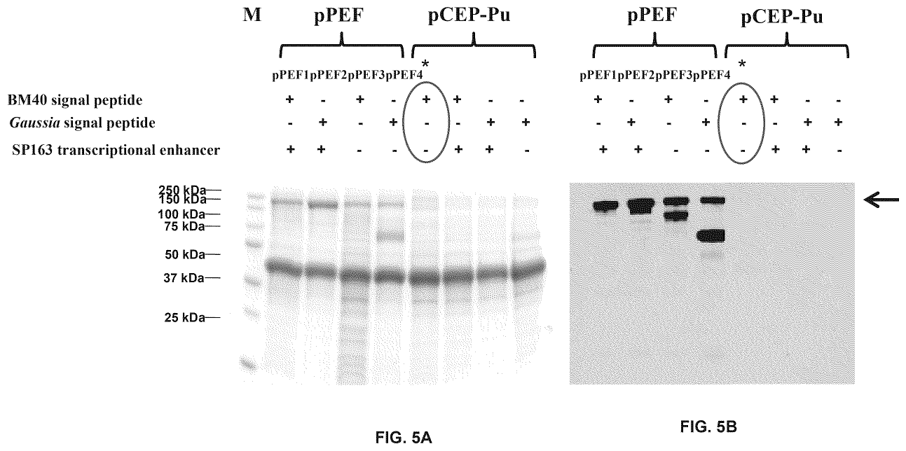

Stable cell lines were created as described above for sVAP-1. It is noteworthy, that as previously, the selection process and the establishment of stable cells for pPEF constructs were a week or two quicker than for the pCEP suite. FIGS. 5A-5B summarize the expression pattern for PLA2R1 in the pPEF and pCEP contexts. PLA2R1 was more highly expressed in the smaller, pPEF, context. Moreover, the highest yield was obtained from the smallest vector tested that contained the Gaussia signal peptide.

The same trend which had been observed for the sVAP-1 has been demonstrated for PLA2R1 as well. Although pPEF4 proves to produce the highest yield, in the case of PLA2R1 the pPEF2 version appears most suitable for a production of full-length protein which is thought to be heavily glycosylated. pPEF vectors therefore allow stable protein expression with high yield in mammalian cells.

### Example 2: Optimisation of the Selective Marker

Once we established that GLuc in the context of pcDNA™4 derived vectors surpasses our usual combination of BM40 signal peptide with pCEP backbone, further attempts were made to better the expression of pPEF2 and pPEF4. In our view these vectors could be made leaner by removing unnecessary regulatory parts such as F1 origin and EM-7 promoter. This would result in smaller vectors thereby increasing transfection efficiency and improving protein yield yet further. The higher initial pool of transfected cells would increase the odds of the construct incorporated into transcriptional hot spots and producing “high expressor” stable cells.

We also wanted to try a different promoter driving the expression of the selective antibiotics' resistance instead of SV40. It was thought to serve two purposes. First, replacing SV40 promoter/origin of replication would make HEK293/T cell line amenable for stable expression. Most of the commercially available plasmids for mammalian expression contain SV40ori which causes genomic instability in cell lines expressing SV40 large T-antigen such as COS7 and HEK293/T (Hunter and Gurney, 1994). This is why HEK293/T cell line is utilised mostly for transient expression making use of its main advantage: a very easy adaptation for growth in suspension which is important for scaling up recombinant protein production. If we could succeed in modifying our vectors and make them suitable for this cell line, then we could achieve a further increase in yield: at the expense of quicker harvests and less labour involved when compared to culturing adherent cells such as HEK293 EBNA1. Second goal could be finding just the right degree of the expression of the resistance gene. Attenuation of selection marker is an important mechanism to create high stringency conditions. It helps bypass a very time-, resource- and labour-consuming process of clonal selection. It is possible now for big biopharmaceutical companies to have a completely automated selection process which is still costly. It is even more tedious for small settings such as individual labs. Transfected cells have to be diluted to obtain individual clones originating from single cells in 24- to 96-well plate formats. Each clone has to be amplified for simultaneous maintenance and screening. Once the screen has revealed the highest expression, the positive clone is chosen for amplification and expansion. However, the attenuated expression of the selective marker can do the perfect job in this regard. It is assumed that a weak promoter can provide just enough expression to confer antibiotic resistance when incorporated into actively transcribed euchromatin, on the provision that the main gene of interest remains intact upon insertion. Thus, only the best (and fewer) expressing cells should survive the selection.

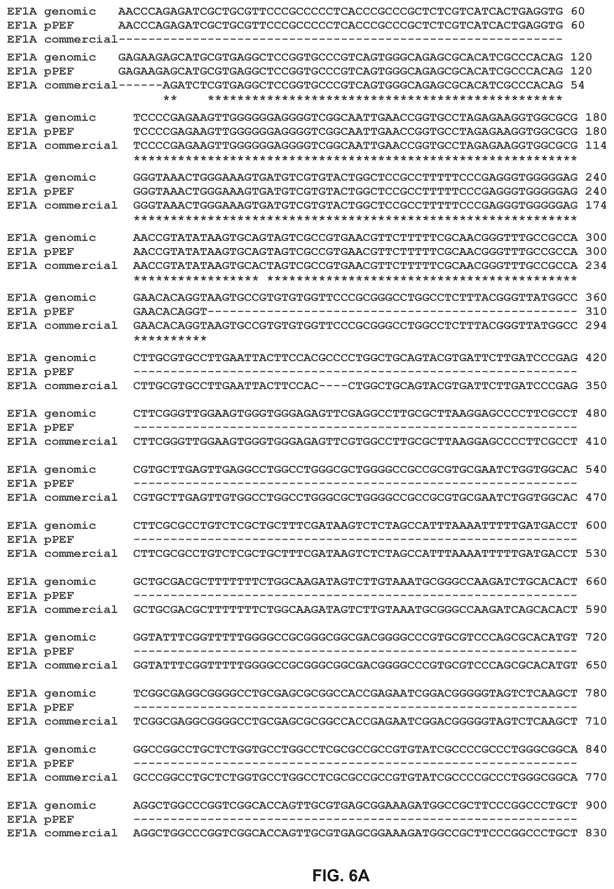

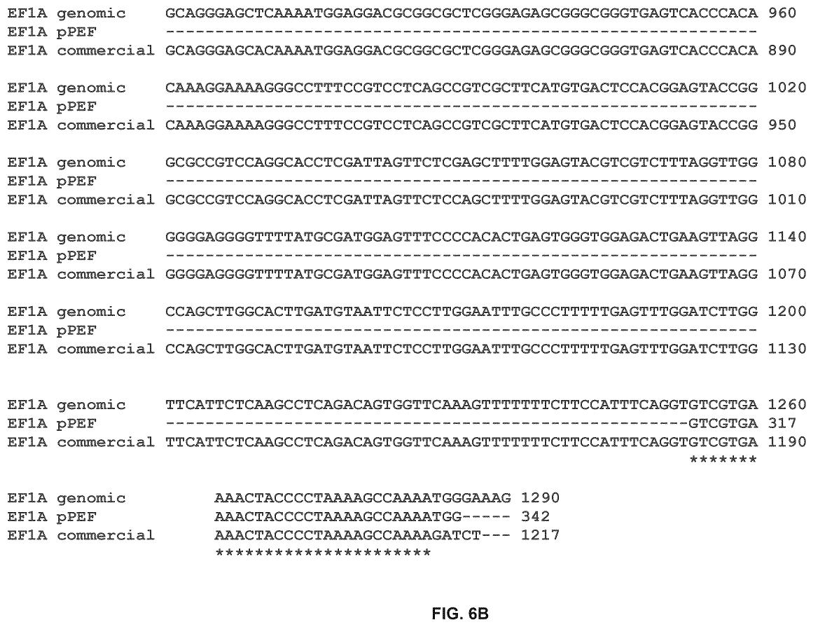

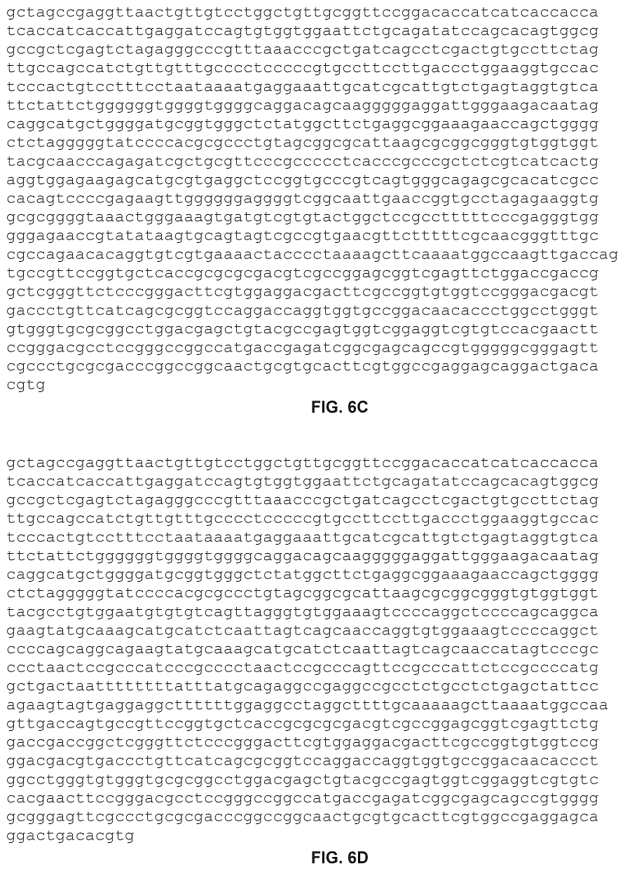

When choosing candidates for the second promoter, we were looking for the ones which would be stronger than or comparable to the SV40. Two constitutive promoters, human elongation factor 1a promoter (EF1A) and chicken b-Actin promoter coupled with CMV early enhancer (CAGG), were reported to occupy intermediate strength position between CMV and SV40 when tested in HEK293/T cells (Qin et al., 2010). We ruled out the CAGG due to its large size, over 1.7 kb, and focused on the EF1A. Commercial vectors mostly contain the larger version of the EF1A promoter, spanning approximately 1.2 kb and include intron 2. Based on the original work of Uetsuki and co-authors (1989), we chose a part of the promoter which starts 73 bp upstream of the commercial EF1A promoter, continues into exon 1, skips the intron 2 and ends at the ATG start codon of exon 2. The total length is 330 bp and represents approximately a quarter of the standard EF1A promoter. We reasoned that the chosen fragment of the full length promoter, or minimal EF1A, should be comparable in strength to the SV40 one, since it contained at least 2 binding sites for Sp1 transcription factor. FIG. 6A represents Clustal multiple alignment of EF1A versions.

With the above considerations in mind, two synthetic constructs were designed: Synthetic fragment I with the minimal EF1A (1206 bp) (see FIG. 6B) and Synthetic fragment II with SV40 promoter (1214 bp) (see FIG. 6C).

Both fragments are flanked by NheI and PmII restriction sites at 5′ and 3′ ends respectively. They incorporate MCS, BGH polyA, EF1A minimal (Fragment I) or SV40 promoter (Fragment II) and Zeocin® antibiotic resistance gene.

To create pPEF5.1 vector, pPEF4 was digested with NheI/PmII and fused with the similarly digested Fragment I. When Fragment II was digested with NheI/PmII and cloned into the prepared pPEF4 vector backbone, it resulted in pPEF5.2 vector. When either fragment was subcloned into the NheI/PmII digested pPEF2 containing SP163 Transcriptional enhancer, then the Max versions of the above mentioned new vectors were produced: either pPEF5.1/Max or pPEF5.2/Max.

To assess whether these more customised vectors are comparable to their predecessors, pPEF2 and pPEF4, in terms of expression levels, we used the same model genes of interest as previously: sVAP and PLA2R1. Additionally, we decided to test the strength of our minimal EF1A promoter. sVAP and PLA2R1 coding sequences were subcloned into pPEF5.1/2 and pPEF5.1/2/Max respectively. HEK293 EBNA1 cell line was transfected with pPEF5.1: sVAP, pPEF5.2: sVAP, pPEF5.1/Max: PLA2R1 and pPEF5.2/Max: PLA2R1. HEK293/T cell line was transfected with pPEF5.1: sVAP and pPEF5.1/Max: PLA2R1.

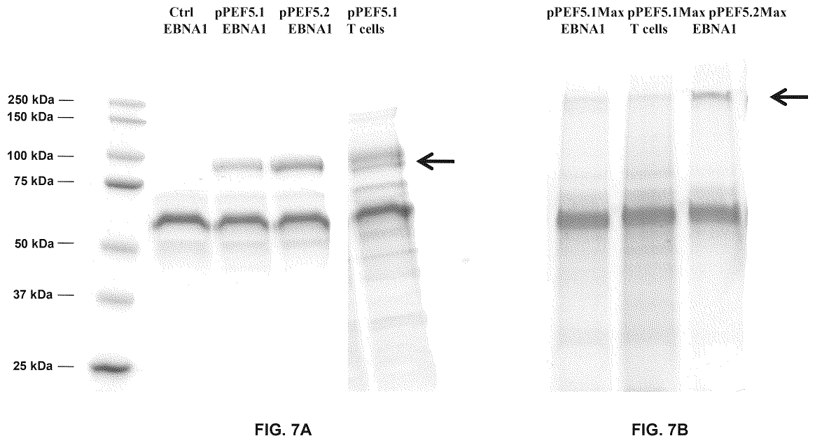

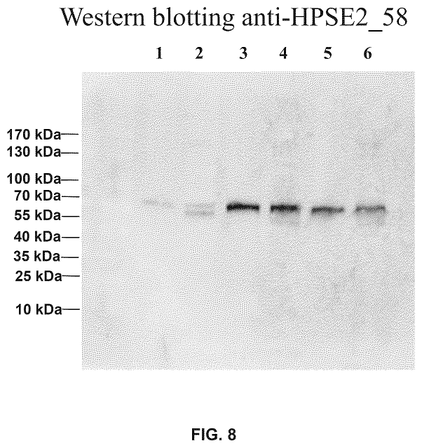

Interestingly, all stable cell lines over-expressing EF1A minimal promoter took 1-2 weeks quicker to establish than their counterparts harbouring SV40 ori/promoter. Also, the initial number of colonies which survived antibiotic selection was significantly higher. FIGS. 7A-7B show the comparison of expression levels between the stable lines and the vectors.

Two important conclusions can be drawn from the experiments:
Firstly, that the EF1A minimal promoter is more active than the SV40 creating less stringent conditions for the selection of the best clones; the SV40 provides the right degree of attenuated expression and therefore should be used for stable line selection unless further work on minimizing EF1A is attempted;
Secondly that pPEF5.2 and pPEF5.2/Max provide comparable if not higher levels of expression than their prototypes, pPEF2 and pPEF4. They should be used for standard protein expression.

### Example 3: Human Vascular Adhesion Protein-1 (VAP-1)

This example demonstrates a robustness of our expression vectors providing a further potential in terms of expression levels for proteins with already high yields.

VAP1 is an endothelial copper-dependent amine oxidase involved in the recruitment and extravasation of leukocytes at sites of inflammation. VAP-1 is an important therapeutic target for several pathological conditions.

The intronless gene that encodes the soluble truncated, form of VAP-1 (sVAP-1; accession No NP 001264660.1; GI: 480306390; residues 29-763) was codon-optimized for expression in human cells and synthesized by Qiagen. The gene was synthesized so that it is flanked at the 5′-end by a NheI restriction site and a BamHI restriction site at the 3′-end, thereby enabling directional subcloning into pCEP-Pu:AC7. The vector contains BM-40 signal peptide followed by NheI which allow extracellular expression of sVAP-1. The construct, as well as all the constructs provided in the Examples section, was DNA sequence verified.

HEK293 EBNA1 cells were transfected with 2 μg of plasmids using the transfection reagent Lipofectamine™ (Invitrogen) according to the manufacturer's instructions. The established HEK293 cells were transferred from the original 6-well plate to a 25 ml culture flask 24 hours post transfection. Antibiotic was added 48 hours post transfection. Stable colonies were pooled together after antibiotic selection for expansion. To harvest media, the cells were seeded into T225 flasks. After reaching cell confluence, the medium was replaced with fresh serum-free medium. Cells were cultured for 7 days followed by harvesting of conditioned medium and replacement with fresh medium. The collected medium, typically 500-550 ml, was centrifuged to precipitate cells and frozen at −20° C. until further use.

For protein purification, CuSO₄ was added to the medium to a final concentration of 0.1 mM, and the medium was incubated at 4° C. for at least 6 h (or on ice overnight). The medium containing sVAP-1 was concentrated to 50 ml using an Amicon stirred ultrafiltration cell fitted with a 30-kDa cut-off filter and subsequently concentrated further to 5 ml using a Vivaspin 20 centrifugal concentrator (Sartorius-Stedim). sVAP-1 was purified by size exclusion chromatography using a HiLoad 26/60 Superdex 200 preparative grade column, pre-equilibrated with 20 mm potassium Pᵢ buffer, pH 7.6, and 150 mm NaCl at 4° C. The same buffer was used throughout the purification procedure, and sVAP-1-containing fractions were pooled, concentrated, and stored at −80° C. until further use.

### Example 4: Human Phospholipase A2 Receptor 1 (PLA2R1)

This example reveals a huge capacity of the pPEF system, especially pPEF5.2/Max version, for difficult/large proteins increasing expression levels 200-fold compared to the standard episomal approach.

PLA2R1 is a major target antigen in idiopathic membranous nephropathy, an organ-specific autoimmune disease. It was also reported to promote the accumulation of reactive oxygen species which induce cell death and senescence implying its role in cancer.

A mammalian codon optimised clone of human PLA2R1 (accession No NP 031392.3; GI: GI: 55953104), covering amino acids 20-1397 which lacks the N-terminal signal peptide and C-terminal transmembrane domain, was synthesized (Genscript®) and included a short C-terminal linker and 10×His affinity tag. The complete insert was recovered from the supplied pUC57 by digestion with NheI and NotI enzymes and ligated into either pCEP-Pu/AC7 or any of the pPEF vectors cut with the same enzymes. HEK293 based stable cell lines were generated as described for sVAP1. PLA2R1 containing media were harvested as above.

Immobilised metal affinity purification included one additional step for pCEP-based stable cell lines prior to loading the media onto AKTAxpress system (GE Healthcare). In view of very low expression level and hence the protein content, the media had to be concentrated approximately 50-fold using a Vivaflow Crossflow 200 (Sartorius Stedim Biotech) or LV Centramate Tangential Flow Filtration System (Pall Corporation). 20 mM Imidazole was added to the media and wash buffer (50 mM NaP, 0.3 M NaCl pH8.0) to increase stringency conditions. The media then was loaded on a 5 ml HisTrap excel column (GE Healthcare) and eluted in an increasing gradient of 500 mM imidazole. The flow-through was re-applied onto the column for the second run due to the fact that some protein was still present in it.

The eluted 2 ml fractions were analysed on a Coomassie stained SDS PAGE and the best fractions were pooled for concentrating and desalting. The desalted protein in the final buffer (PBS or 10 mM Tris, 150 mM NaCl, pH 7.4, 10% Glycerol) was snap frozen in liquid nitrogen and stored at −80° C. until further use.

### Example 5: Expression of HPSE2

This example shows the supremacy of the pPEF vectors compared to the best commercial expression vectors for stable transfection.

Heparanase 2 (HPSE2) is an endoglycosidase that degrades heparin sulfate proteoglycans located on the extracellular matrix (ECM) and cell surface. The protein is involved in remodelling of ECM including angiogenesis and tumor progression. Loss-of-function mutations are strongly linked to urofacial syndrome.

At the time commercial HPSE2 produced in mammalian cells was not available, despite growing interest in fundamental research. Our previous attempts to over-express the protein in the bacterial or insect cells were not very successful in terms of yield and stability. Therefore we attempted to validate our pPEF vector system on this protein.

A complete coding sequence for HPSE2, accession No: AF282887.1; GI: 10801198, was amplified by PCR approach to generate 2 DNA products: 1) the one encoding for a full length protein FL (aa 1-592), b) Δ41 fragment encoding for aa 42-592. Both DNA encoded for 10×His affinity tag at the C-termini. The FL contained its predicted native signal peptide according to SignalP 4.1 software, whereas Δ41 lacked it. The PCR products had introduced NheI and EcoRI flanking sites at 5′ and 3′ ends respectively to allow for a directional cloning into expression vectors. The full-length coding sequence (FL) was cloned into pcDNA™3.1+ (Invitrogen), one of the most standard mammalian expression vectors, which is very similar in terms of vector backbone structure to our pPEF vectors. Both the FL- and Δ41-DNA coding sequences were cloned into NheI/EcoRI digested pPEF4.

The pcDNA™3.1:HPSE2_FL construct was transfected into HEK293 parental cell line since the vector contained a neomycin resistance gene which could not be used in HEK293 EBNA1 cells. The pPEF4:HPSE2_FL and pPEF4:HPSE_Δ41 were transfected into HEK293 EBNA1 cells according to the standard protocol described above.

Serum Free Medium was harvested after 7 days of culturing 100% confluent cells. Since little was known about the degree of secretion from the cells, they were harvested too for cell lysate analysis. FIGS. 7A-7B present a Western blot by a standard Western protocol using specific anti-HPSE2_58 primary sera raised in rabbit.

### Example 6: Recovery of Noggin

This example demonstrates the fact that the pPEF approach can be adapted for the expression of difficult/small targets through the addition of various tags. It is capable of delivering properly processed/folded enzymes the high activity of which is not affected by the presence of the tag. pPEF5.2 appears to perform better than pPEF5.2Max version for over-expression of small to medium sized proteins.

Noggin is an inhibitor of bone morphogenic proteins (BMPs). BMPs play important roles during various stages of development, especially during the establishment of dorsal structures and the development of nervous system. Noggin, together with other co-signalling extracellular factors, exerts correct spatial control of cell fate specification. Apart from its importance for the fundamental research, Noggin is an expensive reagent which is widely used for various cell culture applications. For example, it is used as a supplement for embryonic stem cells to maintain pluripotency or induce cell budding in some specialised cell cultures.

DNA coding for human Noggin accession No NM 005450.4; GI: 189339247 (aa 28-232) lacking its predicted SP and introducing 10×His tag at the C-terminus was codon optimised by GenScript® USA Inc. and cloned into pPEF4 using NheI/EcoRI restriction sites. Newly created stable cell lines did not reveal detectable expression of Noggin by Coomassie staining or Western blotting.

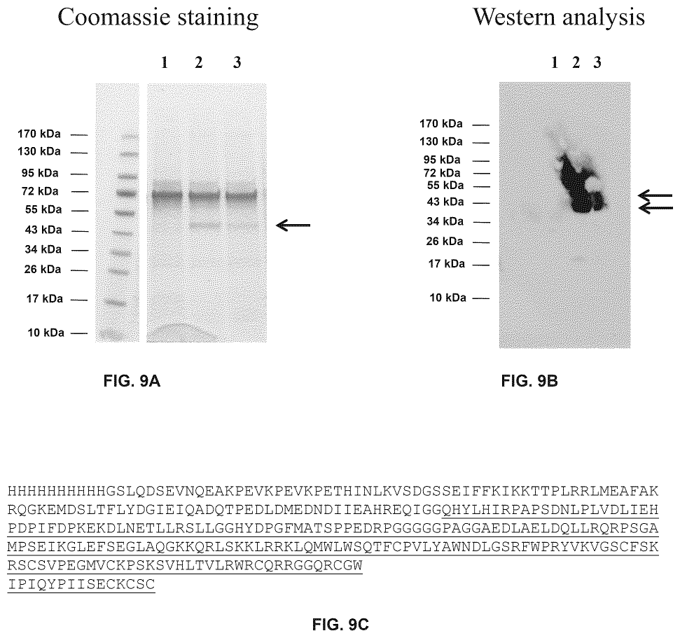

More expression constructs were designed with another version of codon optimised coding sequence by DNA2.0 (USA). This time, two Noggin DNA sequences were made: one coding for the full length FL protein, aa 1-232, thereby incorporating the native SP, and a SUMO-Noggin fusion. The fusion protein (see FIG. 9C) comprised a 10× His tag, the N-terminal SUMOstar secretory tag (LifeSensors), and the mature form of Noggin, aa 28-232 (underlined).

The FL DNA was incorporated into pcDNAT4/HisMax vector using KpnI and EcoRV double digest. The fusion DNA was cloned into pPEF5.2 and pPEF5.2Max via NheI and NotI restriction sites.

Stable cell lines were created as described above and analysed for the expression of either FL Noggin or Sumo-Noggin fusion. No detectable expression level of FL Noggin were found in the stable lines transfected with pcDNA™4/HisMax: Noggin vector. FIG. 9 presents Coomassie staining and Western blotting of SDS PAGE of Sumo-Noggin fusions in pPEF5.2 and pPEF5.2/Max. As one can see, there is a higher expression level of the fusion protein in pPEF5.2 vector than in its Max version.

For scale-up production, 1 L of Sumo-Noggin-containing serum-free media were harvested and loaded (cell-free) onto a 5 ml HisTrap Excel column for Affinity Chromatography purification using AKTAxpress (GE Healthcare) as described above.

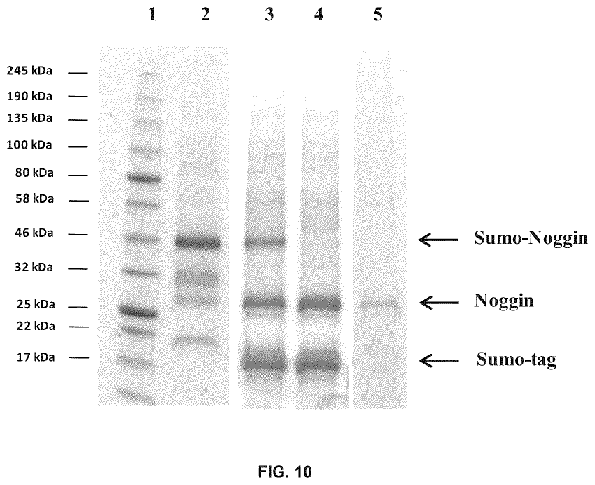

The purified Sumo-Noggin fusion batch was divided into two halves: first half was left intact as a fusion, the second one was intended for Sumo-tag removal. The tag removal required some optimisation, since the manufacturer's recommended incubation temperature, up to 30° C., did not produce a fully cleaved Noggin. Instead, the following optimised conditions were established: 1U of SUMOstar protease (LifeSensors, Tebu-Bio) per 90 μg of the substrate for 1.5 hours at 40° C. FIG. 10 demonstrates the integrity of Noggin protein after the tag digest at such a relatively high temperature. This noteworthy stability testifies to a very efficient tight folding of the recombinant molecule despite the large tag.

To recover the cleaved Noggin, the fully digested fusion protein was incubated with Ni-NTA Agarose resin (Qiagen) at 4° C. to bind the cleaved Sumo-tag and SUMOstar protease (both are His-tagged). The unbound Noggin was collected and quantified (lane 5 on FIG. 9). The identity of Noggin was confirmed by Mass Spectroscopy analysis.

In activity assays in organoid cultures both proteins were found to be more enzymatically active than their commercial analogue, recombinant murine Noggin (PeproTech, Cat No 250-38).

### Example 7: Expression of RET (Contains a Transmembrane Domain)

This example exhibits the suitability of pPEF for the expression of membrane proteins which are notorious for their toxic effect on the cell when over-expressed. This opens up an opportunity to use the vectors not only for recombinant protein production but also for drugs screen.

Rearranged during transfection (RET) is a proto-oncogene. It is a receptor which encodes for the signalling component of the glial cell line-derived neutrotrophic factor-family ligands receptor. This receptor tyrosine kinase is essential for spermatogenesis, development of the sensory, sympathetic, parasympathetic, and enteric nervous systems and the kidneys, as well as for maintenance of adult midbrain dopaminergic neurons. A number of loss-of-function mutations have been identified throughout the RET gene that lead to Hirschsprung disease, a congenital disorder characterized by a loss of enteric neurons in the distal portions of the colon and small intestine. Conversely, mutations that result in constitutively active receptors have been linked to tumors of various neuroendocrine tissues, including the thyroid, parathyroid, and adrenal glands. Some forms of RET have been used for high-throughput drug screens at the Cancer Research UK Manchester Institute, formerly the Paterson Institute for Cancer Research. Some short RET domains/regions are available in recombinant form commercially due to a huge interest in cancer research, but not long forms. It was chosen for our validation studies as a good example of membrane-associated receptor. Membrane bound proteins are thought to share the same metabolic pathway with the secreted proteins. On the other hand, over-expression of anchored proteins tends to have toxic effect due to cellular metabolic overburdening.

Human RET cDNA gene clone (cat: HG 11997-G) was ordered from Stratech Scientific Ltd. A RET51 cDNA form coding for aa 30-1114 (lacking its predicted signal peptide) and the C-terminal 10×His tag was amplified by PCR and cloned into pPEF5.1Max using NheI/NotI restriction sites.

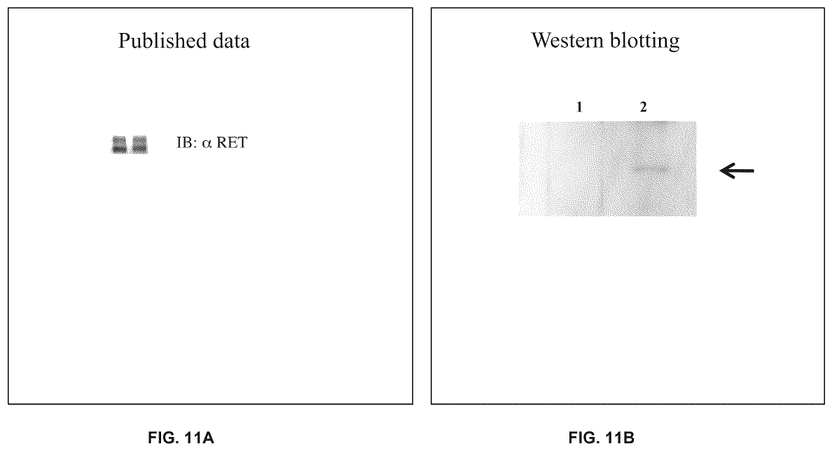

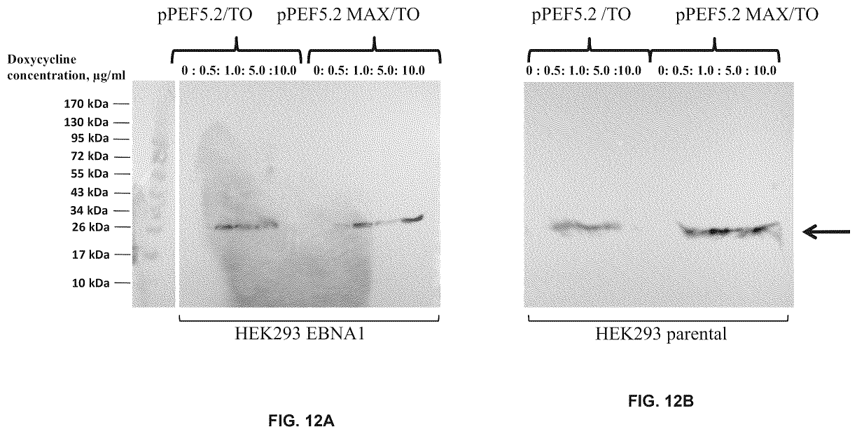

RET51 stably expressing cells were harvested, lysed and analysed for RET expression by Western blotting technique as shown on FIGS. 11A-11B.

### Example 8: Matrix Metalloproteinase 12 (MMP-12)

This example shows the pPEF vectors as the robust tool to over-express difficult/toxic targets through inducible promoter modifications. It also demonstrates comparable performance in different HEK293-derived cell lines.

MMP12 is also known as macrophage elastase. It is a member of the matrix metalloproteinases. MMPs are a family of structurally related, zinc-containing enzymes that degrade the ECM and connective tissue proteins. The proteolytic effects of MMPs play an important role in vascular remodeling, cellular migration and the processing of ECM proteins and adhesion molecules, hence their huge implications for cancer and arthritis research. No wonder that this group of enzymes has been a pharmaceutical target for nearly 30 years, yet none of the developed drugs has successfully passed clinical trials. One of the main reasons for the failure—the complexity of MMPs regulation at various levels and their implication in a multitude of pathways. For example, in addition to MMP-12 diverse roles in elastolysis during uterine remodelling, tissue remodeling in inflammatory respiratory diseases such as chronic obstructive pulmonary diseases (COPD), elastin degradation in atherosclerotic lesions as well as lung cancer, esophageal squamous cell carcinoma, and skin cancer, it has been recently reported as a transcriptional factor conferring antiviral immunity (Marchant et al., 20014).

MMP-12 (accession No NP 002417.2) is synthesized as a 56 kD precursor molecule consisting of a propeptide (aa 17-60), catalytic (aa 106-263) and hemopexin (aa 277-470) domains connected via a short linker. One of the astounding properties of this enzyme is that it catalyses its own cleavage (auto-catalysis): once an active form is produced by yet an unknown mechanism, MMP-12 undergoes a cascade of cleaving events producing intermediate short-lived forms until the separate catalytic and hemopexin domains produced. Available commercial forms of recombinant MMP-12 are mostly produced in E. coli lacking specific activity. The only available enzymatically active recombinant MMP-12 is supplied as a 10 μg latent precursor which needs to be activated in vitro (R&D Systems, Cat No 917-MP-010).

Our initial efforts to stably over-express active form, A105 MMP-12 (aa 106-470) in pPEF4 and pPEF5.2Max failed. Intriguingly, repeated transfections produced the same scenario: initial small colonies, which always look a promising sign for an emerging cell line, would disappear after few days of culturing. We hypothesised that the increasing levels of active MMP-12 secreted into the media should degrade ECM thereby detaching stably expressing cells and eroding establishing colonies. The positive cells would be removed with old media.

This is why a stable cell line could never be established. To test our hypothesis we performed transient transfections which confirmed our reasoning: Western analysis revealed the presence of MMP-12 72 hours post transfections. To adopt new approaches, we modified our expression vectors for inducible expression (approach 1), while also trying to over-express MMP-12 precursor (aa 17-470) in the standard pPEF5.2 with the use of specific inhibitors (approach 2).

For the first approach, a doxycycline inducible expression, we adopted the principle described for T-REX™ system developed by Life Technologies (tools.lifetechnologies.com). Firstly, HEK293 parental and HEK293 EBNA1 cell lines which would stably express tetracycline repressor (TetR) had to be generated. It was done by transfecting pcDNA™6/TR vector (Life Technologies) into these lines. Stable lines were selected by their resistance to blasticidin. Secondly, to introduce two tetracycline operator 2 (TetO₂) sites into the Pcmv promoter of our expression vectors, we digested pcDNA™5/FRT/TO vector (Life Technologies) with NdeI/KpnI. When we swapped the excised DNA fragment with the one from similarly digested pPEF5.2 and pPEF5.2/Max, we obtained the doxycycline (a more stable variant of tetracycline) inducible versions, pPEF5.2/TO and pPEF5.2/Max/TO respectively.

DNA sequence coding for the active form of MMP-12 (aa 106-470) was cloned into doxycycline inducible versions of pPEF, pPEF5.2/TO and pPEF5.2Max/TO, through NheI/NotI digest. Both constructs were transfected into HEK293 parental and HEK293 EBNA1 cell lines. When analysed for the expression levels, all 4 construct: cell line combinations demonstrated comparable levels of A105 MMP-12 expression when induced with doxycycline (FIGS. 12A-12B).

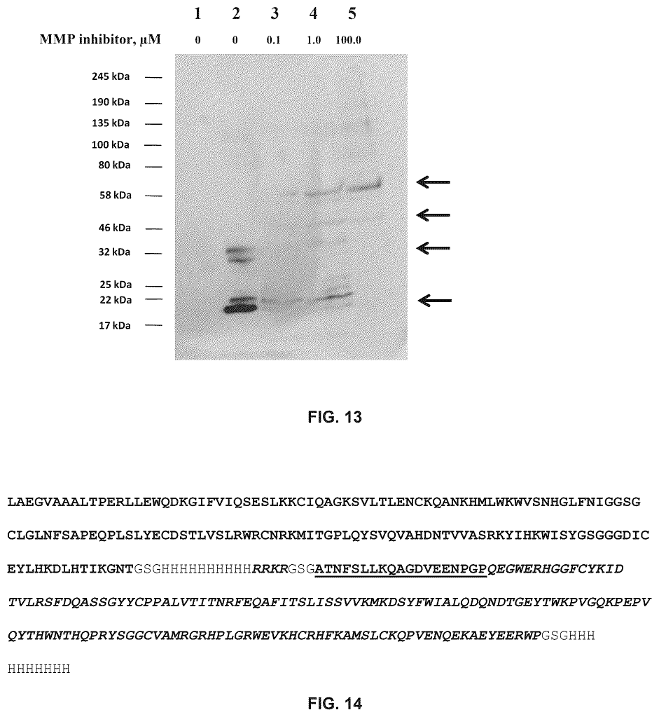

The second approach, the over-expression of MMP-12 precursor in pPEF5.2Max with inhibitor's treatment (Z-Pro-Leu-Gly-NHOH broad range MMP inhibitor from Enzo Life Sciences), was also successful as demonstrated by FIG. 13. The propeptide form could be produced and “preserved” by blocking its further processing and auto-catalysis.

### Example 9: Purification of Ricin Domain of PLA2R1

This example demonstrates high efficiency of pPEF to deliver small proteins via bicistronic modifications.

Initially we planned to over-express 2 domains of PLA2R1: Ricin and C-type lectin domain 3 (C3). Our previous experience showed that the mammalian HEK293-based expression system is not suitable for the production of small proteins which are below 40 kDa threshold. This might explain the failed initial attempts to over-express Noggin. This is why in the case of Ricin domain of PLA2R1 we looked for alternatives. One way to overcome the problem would be the attachment of a bulky tag such as Sumo. However, the disadvantage of this method is the need in additional steps aimed at tag removal and the recovery of a tag-less protein. It is costly, as well as time- and labour-consuming, delivering the protein of interest in the amounts which are a fraction of the initial yield. Therefore we decided to try a 2A approach instead (Luke, 2012) which would allow a simultaneous expression of two proteins driven by the same promoter. The principle is based on the insertion of the 2A-like viral sequences used to mediate protein cleavage from a single open reading frame through a ribosomal skip mechanism. We used a version of 2A sequence derived from porcine teschovirus-1 (P2A) as one of the most efficient and robust (Kim et al., 2011), delivering co-expressed proteins in the stoichiometry 1:1. The designed sequence is presented in FIG. 14. While the bicistronic P2A motif should allow for a simultaneous expression of the two domains (both with the C-terminal 10×His tags), the introduced Furin cleavage site should ensure the cleavage of the P2A motif from the Ricin domain by the endogenous Furin upon its secretion. It was expected that the C3 domain would be secreted too, following the signal peptide of the Ricin domain.

The DNA sequence encoding for the fragment was cloned into pPEF5.2 expression vector and a stable cell line was generated as described above.

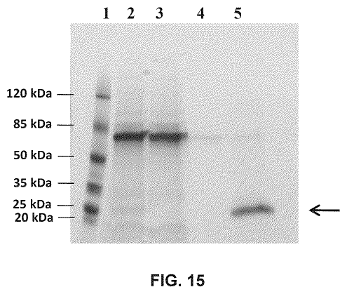

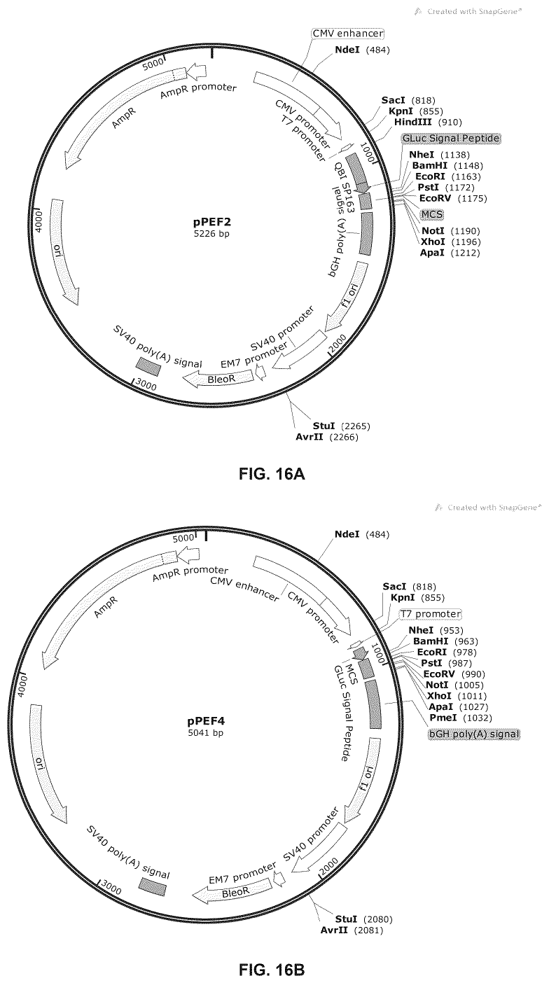

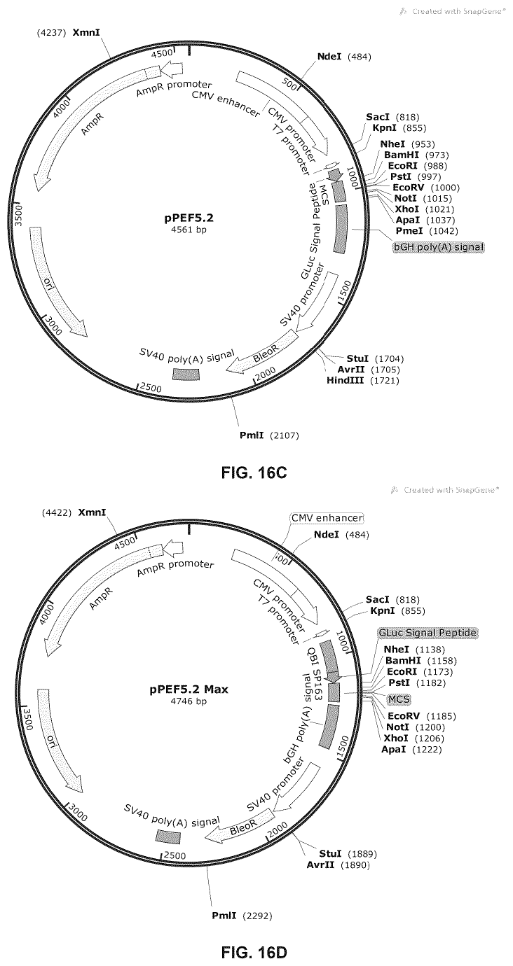

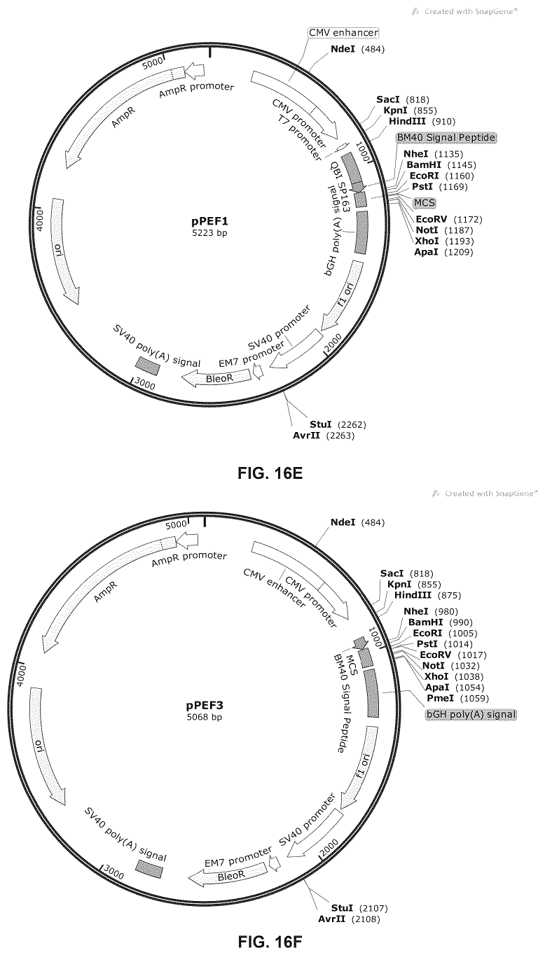

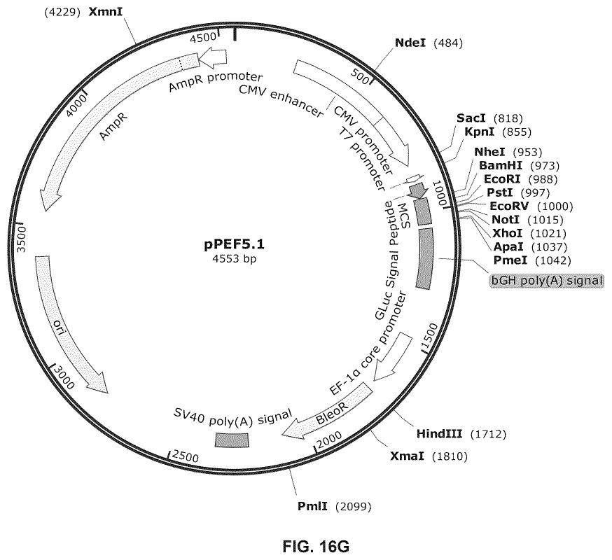

Coomassie staining and Western blotting of the Ricin-C3 P2A conditioned media revealed the presence of positive band of approximately 25 kDa (FIG. 15, lane 2). Since the observed band could account for either histidine-tagged protein's expected size, it was unclear whether Ricin and C3 domains are expressed simultaneously.

To ascertain the presence of both recombinant proteins, small scale manual batch purification was performed. 16 ml conditioned media supplemented with 20 mM Imidazole, was incubated with pre-washed Ni-NTA agarose (QIAGEN) for 1 hour at room temperature.

The single washing step was performed in 50 mM NaP, 0.3 M NaCl PH 8.0, 50 mM Imidazole. The bound protein was eluted in the same buffer containing 500 mM Imidazole.

After Coomassie analysis of all the purification steps (FIG. 15), the eluted protein was buffer exchanged into 10 mM Bis Tris, 150 mM NaCl, pH 7.4 10% Glycerol and snap frozen. The eluted band was analysed by Mass Spectrometry which detected the presence of Ricin domain only. Apparently, the addition of a signal peptide is an important prerequisite for the efficient secretion of the second protein when using the 2A approach.

### Example 10: Strategy to Remove Lumio and 10×His Tag from MCS of pPEF5.2 to Produce pPEF5

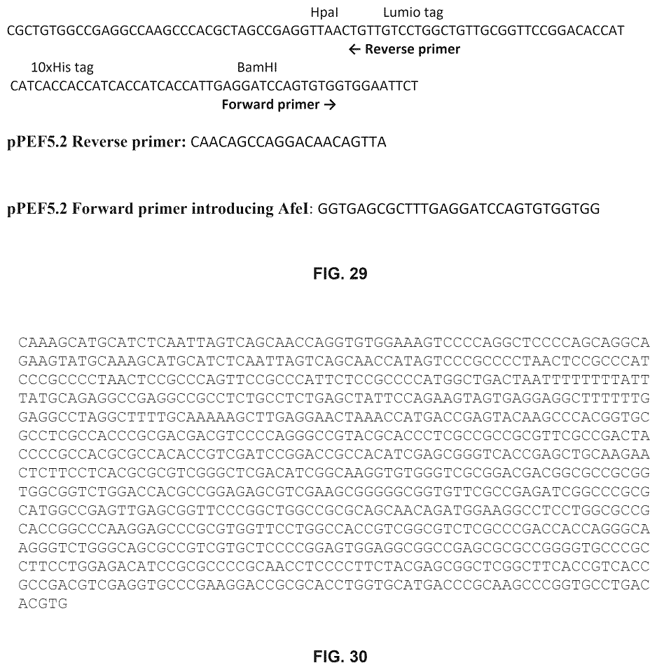

In order to further reduce the size of the size of the vector, we removed the Lumio and 10×His tag sequences from the MCS. Primers were designed as shown in FIG. 29. pPEF5.2 plasmid was amplified via a PCR approach using the above primers. The resulting PCR product was digested with HpaI and AfeI and blunt ligated to produce a pPEF5 plasmid.

### Example 11: Strategy to Replace Zeocin® Antibiotic Resistance Gene in pPEF5 with Puromycin to Produce pPEF5PuroR

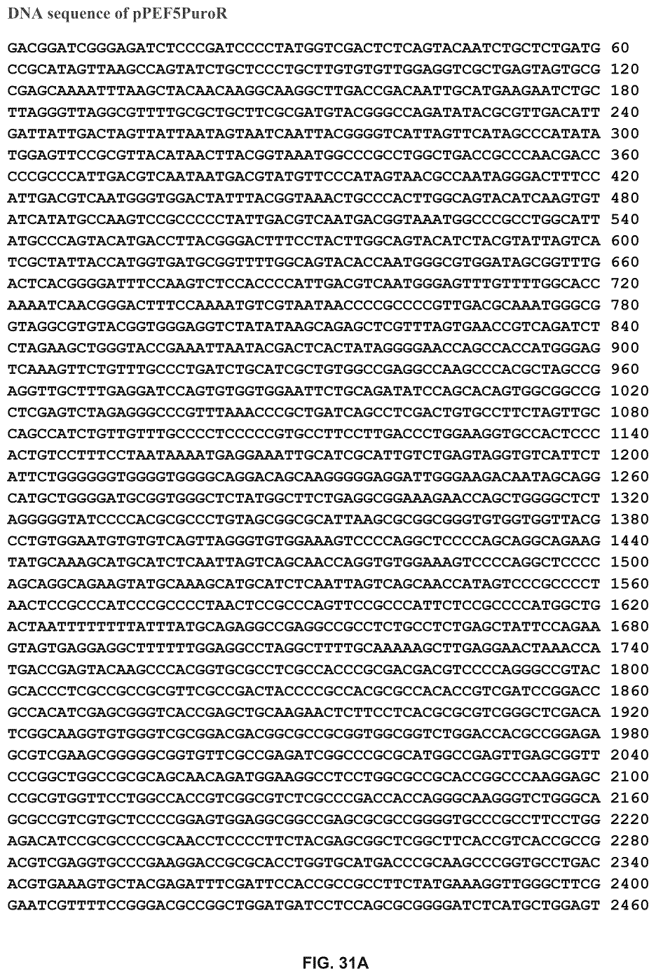

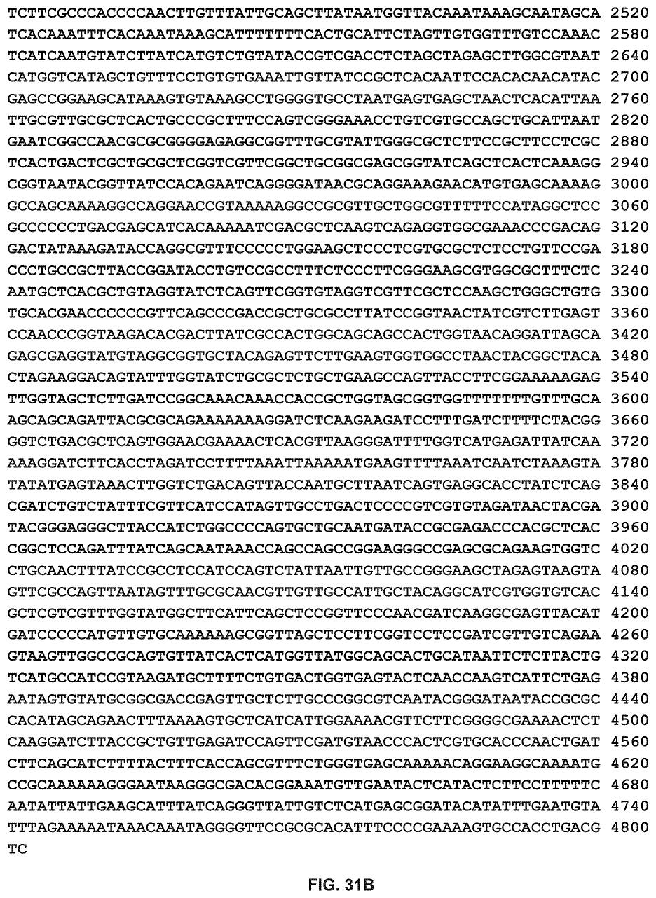

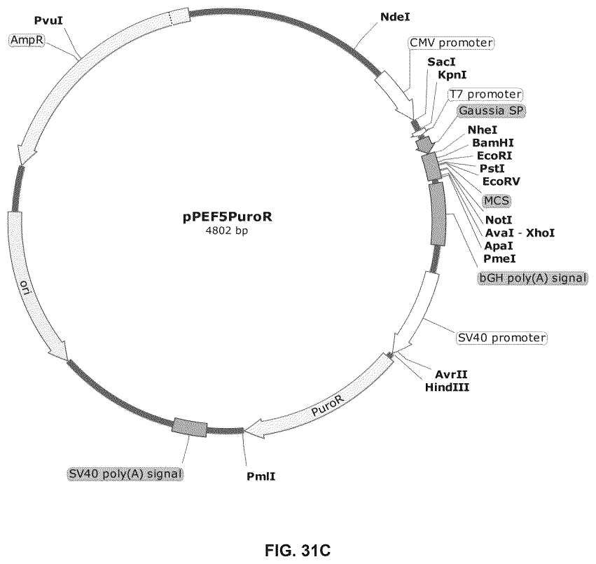

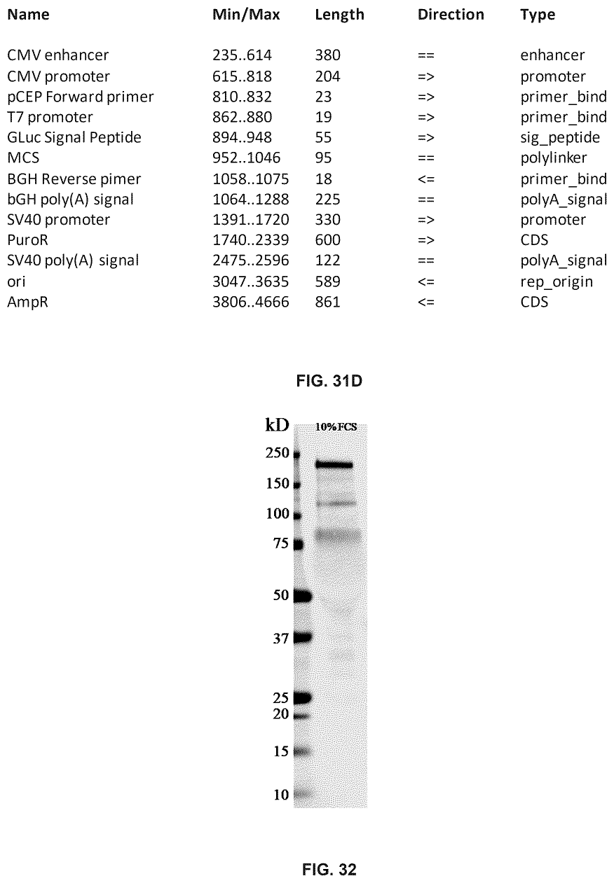

Synthetic PuroR DNA sequence with internal HindIII and PmII restriction sites was commissioned from GenScript® (see FIG. 30). pPEF5 was digested with HindIII and PmII to remove the DNA fragment coding for Zeocin® antibiotic resistance and replace it with HindIII/PmII digested synthetic DNA coding for Puromycin resistance. The resulting vector was named pPEF5PuroR (shown in FIGS. 31A-31D).

### Example 12: Human Secreted Thrombospondin Type-1 Domain-Containing Protein 7A (THSD7A)—aa 48-1606

The protein is found almost exclusively in endothelial cells from placenta and umbilical cord. The encoded protein appears to interact with alpha (V) beta (3) integrin and paxillin to inhibit endothelial cell migration and tube formation. This protein may be involved in cytoskeletal organization. Variations in this gene may be associated with low bone mineral density in osteoporosis.

A synthetic DNA fragment coding for codon optimised soluble form of THSD7A with 10×His tag at the C-terminus was ordered from GenScript®. The DNA was subcloned into NheI/BamHI digested pPEF5PuroR. HEK293 EBNA1 cells were transfected with 2 μg of THSD7A constructs using Lipofectamine 2000 transfection reagent (Life Technologies) according to the manufacturer's instructions. Stable cell lines were established after 6 weeks of selection. A Western blotting analysis of the complete conditioned media demonstrated THSD7A expression is shown in FIG. 32.

### Example 13: Human Secreted Amyloid Precursor Protein (APP) α (Aa 18-612) and β (Aa 18-596) Forms

APP is a single-pass transmembrane protein expressed at high levels in the brain and metabolized in a rapid and highly complex fashion by a series of sequential proteases. Research suggests that physiologic generation of the neurotoxic Aβ peptide from sequential APP proteolysis is the crucial step in the development of Alzheimer's Disease (AD) which is the leading cause of dementia worldwide, is characterized by the accumulation of the β-amyloid peptide (Aβ) within the brain.

Corresponding DNA fragments coding for α and β forms of APP were PCR amplified from the APP mRNA transcript (GenBank accession No: NM 201414.2). The PCR products had introduced flanking NheI and NotI restriction sites. Both encoding DNAs were subcloned into pPEF5PuroR.

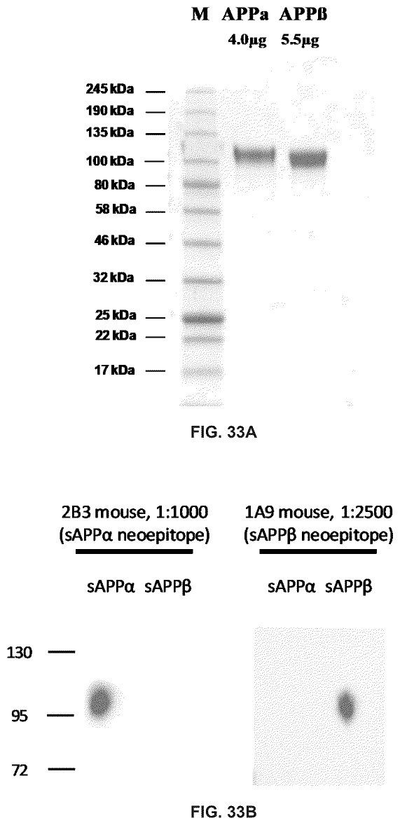

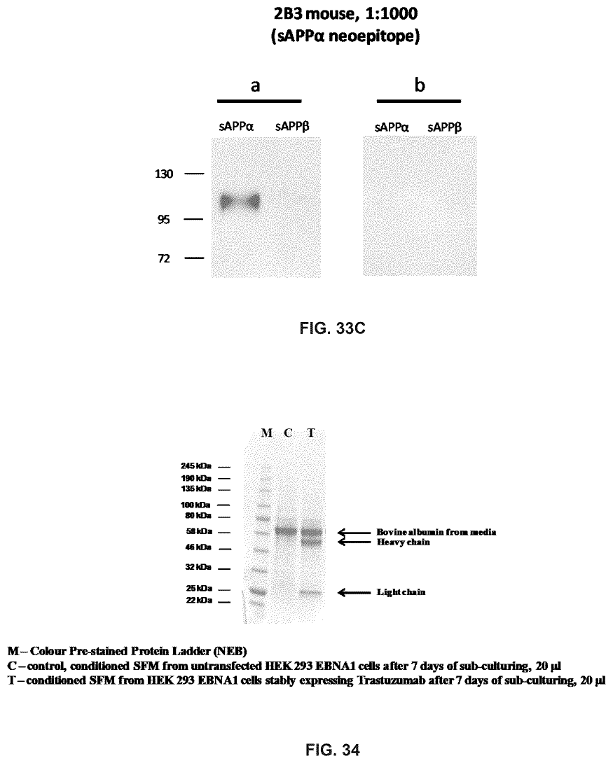

Transfection and creation of APPα and APP β stable cell lines were carried out according to the established protocol described above. Media from both stable cell lines were harvested for manual protein purification (batch method) as described for the Example No 9 in our Specifications document. Aliquots of the purified protein were analysed by Coomassie staining for protein integrity and purity. See FIG. 33A.

Further analysis with isoform-specific neoepitope antibodies (antibodies which recognise a specific C-terminal amino acid) (See FIG. 33B) and comparison of the purified proteins with their commercial analogues demonstrated their superior qualities (see FIG. 33C).

### Example 14: Trastuzumab (TZM)

Trastuzumab represents a distinct class or recombinant proteins, monoclonal recombinant antibodies or mAbs. The recombinant IgG1 kappa, humanized monoclonal antibody that selectively binds with high affinity in a cell-based assay to the extracellular domain of the human epidermal growth factor receptor protein, is an extremely popular therapeutic. It is used to treat breast cancer.

The DNA sequences of Heavy and Light chains of TZM were obtained from DrugBank (Accession Number DB00072).

The synthetic DNA sequence coding for codon optimised Heavy and Light chains of Trastuzumab connected via a P2A sequence (as described for the Example 9 in the original Specifications document) and with NheI and BamHI flanking sites was ordered from GenScript®.

The DNA was digested with NheI/BamHI and subcloned into pPEF5PuroR.

HEK293 EBNA1 cells were transfected according to the standard protocol. TZM expressing stable cell line was established after 4 weeks of selection. Coomassie staining of conditioned SFM showed high expression levels of Trastuzumab is shown in FIG. 34.

### Example 15: Validation of the Expression System in CHO-K1 Cells

### A: The Expression of Trastuzumab (TZM) in CHO-K1 Cells.

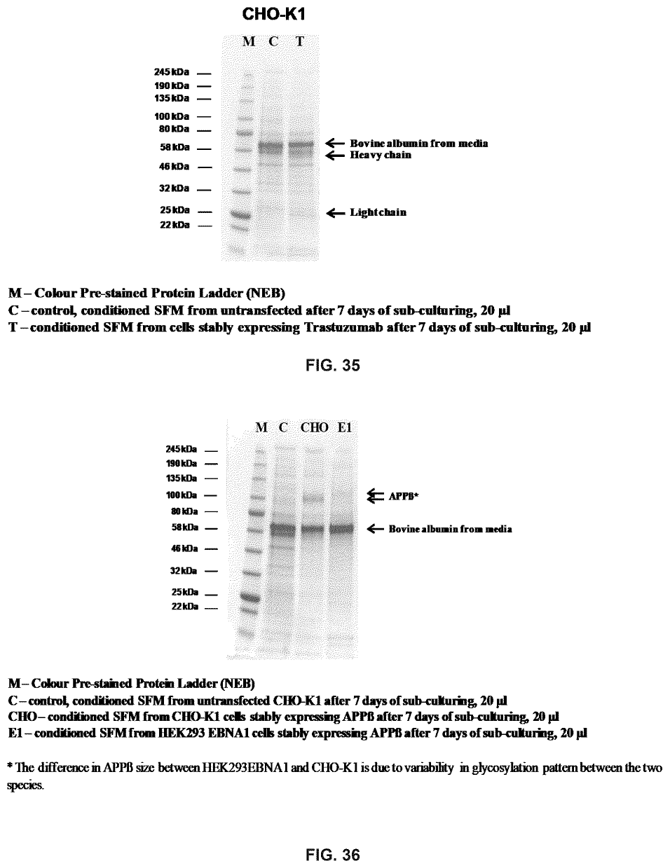

The above TZM expression construct was transfected into CHO-K1 cell line, using the same protocol as for HEK293 EBNA1. Stable CHO-K1 cell lines were established after 4 weeks of antibiotic selection. Coomassie staining of the conditioned SFM media revealed a perceptible TZM expression, although lower than in HEK293 EBNA1 cells (see FIG. 35).

### B: The Expression of Human Secreted Amyloid Precursor Protein Beta Form (APPβ) in CHO-K1 Cells.

APPβ expression construct was transfected into CHO-K1 cells and established stable cell lines were analysed for APPB expression levels. Comparative Coomassie staining revealed a higher expression level of the protein in CHO-K1 cells than in the stable HEK293 EBNA 1 (see FIG. 36).

### Example 16: Establishment of a Minimal EF1A Promoter

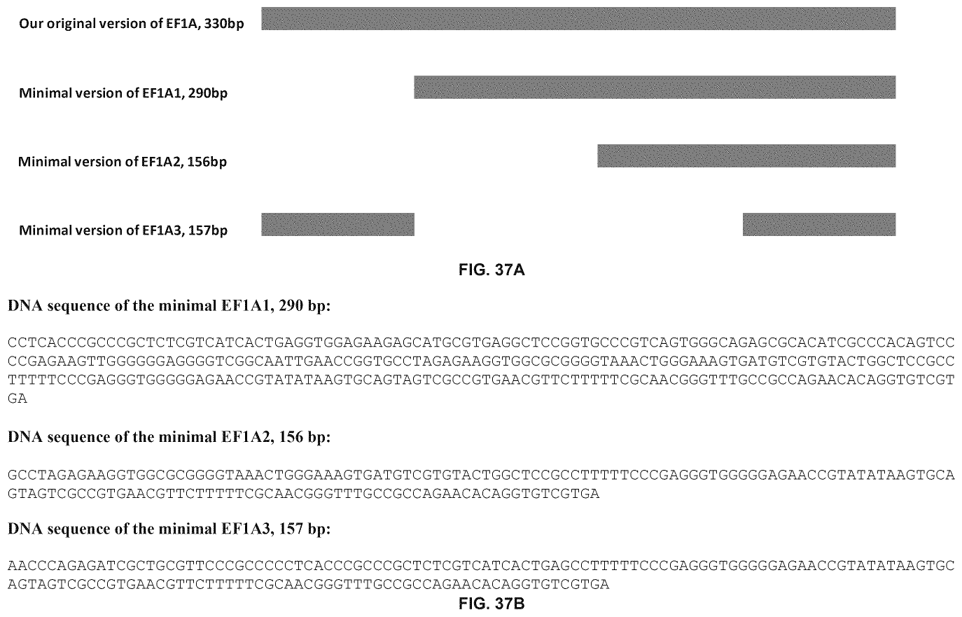

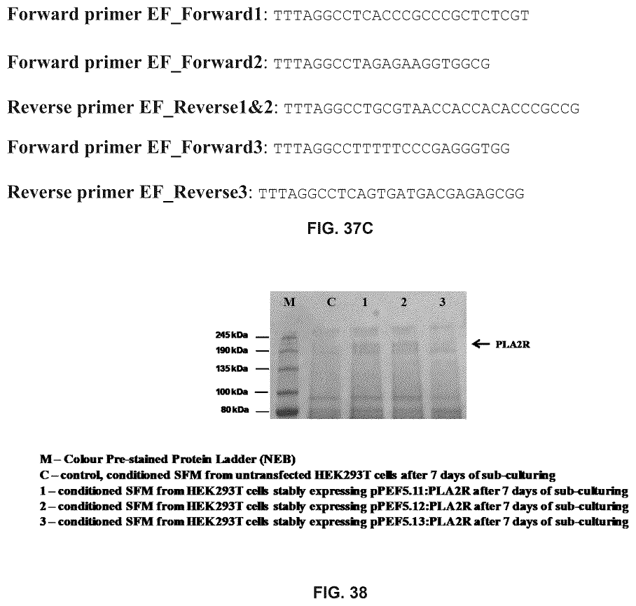

In Example 2 we developed a short version of EF1A promoter. However, despite representing only a quarter of length of the traditionally used EF1A promoter, it still demonstrated elevated activity when compared with the SV40 one. This higher strength of expression is not desirable when governing a selectable marker since it reduces the stringency of selection. Further attempts to attenuate promoter activity are reduce its size have been undertaken with the strategy described below. The constructs are shown schematically in FIG. 37A and their sequences provided in FIG. 37B.

Primers were designed to delete regions from the 5′ end of the original minimal EF1A promoter in pPEF5.1 vector (all the primers introduced a StuI restriction site). Sequences are given in FIG. 37C.

pPEF5.1 vector was used as a template to create shorter versions of EF1A promoter via a PCR approach using the above primers. The generated PCR products were digested with StuI and self-ligated to produce pPEF5.11, pPEF5.12 and pPEF5.13 respectively. All three vectors were digested with NheI and NotI to accommodate the same PLA2R gene as described in the original Specification document. pPEF5.11:PLA2R, pPEF5.12:PLA2R and pPEF5.13:PLA2R constructs were transfected into HEK293T cell line as described previously. Stable cell lines harbouring pPEF5.11:PLA2R and pPEF5.12:PLA2R were established much quicker than the one over-expressing pPEF5.13:PLA2R. The latter cell line was formed from very few cells which survived antibiotic selection. Coomassie staining analysis of the conditioned media from all 3 stable cell lines demonstrated a comparable expression level of PLA2R for pPEF5.11 and pPEF5.12 and no detectable expression for pPEF5.13 (see FIG. 38).

It appears that the third version of the minimal EF1A promoter, EF1A3, is non-functional. The other two versions, EF1A1 and EF1A2, showed a similar expression level in HEK293T cells. It is recommended to test their strength in HEK293 EBNA1 line. It is possible, that although EF1A2 version has a greater potential for increased selection stringency, it could not be achieved in HEK293T due to some intrinsic metabolic bottlenecks. EBNA1 cell line has always proved to be the best cell line in terms of high expression levels when the same construct was expressed in HEK293 parental, HEK293 EBNA1 and HEK293T derivatives. Therefore, it can be assumed, that once placed in the genomic and metabolic context of this host cell line, EF1A2 minimal promoter can execute a better selection, hence a stronger drive for high-expressing stable clones, when compared to EF1A1.

### REFERENCES

Durocher, Y., Perret, S. and A. Kamen. High-Level And High-Throughput Recombinant Protein Production By Transient Transfection Of Suspension-Growing Human 293-EBNA1 Cells. Nucleic Acid Research, Vol. 30, No 2, pp 1-9, 2002.
Tannous, B. A., Kim, D-E., Fernandez, J. L., Weissleder, R. and X. O. Breakefield. Codon-Optimised Gaussia Luciferase cDNA For Mammalian Gene Expression In Culture And In Vivo. Molecular Therapy, Vol. 11, No 3, pp 435-443, 2005.
Knappskog, S., Ravneberg, H., Gjerdrum, C., Tröβe, C., Stern, B. and I. F. Pryme. The Level Of Synthesis And Secretion Of Gaussia princeps Luciferase In Transfected CHO Cells Is Heavily Dependent On The Choice Of Signal Peptide. Journal of Biotechnology, No 128, pp 705-715, 2007.
Stern. B., Olsen, L. C., Tröβe, C., Ravneberg, H. and I. F. Pryme. Improving Mammalian Cell Factories: The Selection Of Signal Peptide Has a Major Impact On Recombinant Protein Synthesis And Secretion In Mammalian Cells. Trends In Cell And Molecular Biology, No 2, pp 1-17, 2007.
Wen, B., Deng, Y., Guan, J., Yan, W., Wang, Y., Tan, W. and J. Gao. Signal Peptide Replacements Enhance Expression And Secretion Of Hepatatis C Virus Envelope Glycoproteins. Acta Biochimica Et Biophysica Sinica, Vol. 43, No 2, pp 96-102, 2011.
Swaroop, A., Hogan, B. L. M. and U. Franke. Molecular Analysis Of The cDNA For Human SPARC/Osteonectin/BM-40: Sequence, Expression, And Localisation Of The Gene To Chromosome 5q31-q33. Genomics, No 2, pp 37-47, 1988.
Jean, F., Stella, K., Thomas, L., Liu, G., Xiang, Y., Reason, A. J. and G. Thomas. a1-Antitrypsin Portland, A Bioengineered Serpin Highly Selective For Furin: Application As An Antipathogenic Agent. Proceedings Of The National Academy Of Sciences USA, Vol. 95, pp 7293-7298, 1998.
Bornkamm, G. W., Berens, C., Kuklik-Roos, C., Bechet, J-M., Laux, G., Bachl, J., Korndoerfer, M., Schlee, M., Hölzel, M., Malamoussi, A., Chapman, R. D., Nimmerjahn, F., Mautner, J., Hillen, W., Bujard, H. and J. Feuillard. Stringent Doxycycline-Dependent Control Of Gene Activities Using An Episomal One-Vector System. Nucleic Acid Research, Vol. 33, No 16, e137, pp 1-11, 2005.
Kohfeldt, E., Maurer, P., Vannahme, C. and R. Timpl. Properties Of The Extracellular Calcium Binding Module Of The Proteoglycan Testican. FEBS Letters, Vol. 414, No 3, pp 557-561, 1997.
Heuts, D. P. H. M., Gummadova, J. O., Pang, J., Rigby, S. E. J. and N. S. Scrutton. Reaction Of Vascular Adhesion Protein-1 (VAP-1) With Primary Amines: Mechanistic Insights From Isotope Effects And Quantitative Structure-Activity Relationships. The Journal Of Biological Chemistry, Vol. 286, No 34, pp 29584-29593, 2011.
Kanigicherla, D., Gummadova, J., Mckenzie, E. A., Roberts, S. A., Harris, S., Nikam, M., Poulton, K., McWilliam, L., Short, C. D., Venning, M. and P. E. Brenchley. Anti-PLA2R1 Antibodies Measured By ELISA Predict Long-Term Outcome In A Prevalent Population Of Patients With Idiopathic Membranous Nephropathy. Kidney International, No 5, pp 940-948, 2013.
Hunter, D. J. and E. G. Gurney. The Genomic Instability Associated With Integrated Simian Virus 40 DNA Is Dependent On The Origin Of Replication And Early Control Region. Journal Of Virology, Vol. 68, No 2, pp 787-796, 1994.
Qin, J. Y., Zhang, L., Clift, K. L., Hulur, I., Xiang, A. P., Ren, B-Z. and B. T. Lahn. Systematic Comparison Of Constitutive Promoters And The Doxycycline-Inducible Promoter. PLOS ONE, Vol. 5, No 5, e10611, 2010.
Uetsuki, T., Naito, A., Nagata, S. and Y. Kaziro. Isolation And Characterization Of The Human Chromosomal Gene For Polypeptide Chain Elongation Factor-1a. The Journal Of Biological Chemistry, Vol. 264, No 10, pp 5791-5798, 1989.
Öhman, J., Jakobsson, E., Källström, U., Elmblad, A., Ansari, A., Kalderén, C., Robertson, E., Danielsson, E., Gustavsson, A-L., Varadi, A., Ekblom, J., Holmgren, E., Doverskog, M., Abrahmsen, L. and J. Nilsson. Production Of a Truncated Soluble Human Semicarbazide-Sensitive Amine Oxidase Mediated By a GST-Fusion Protein Secreted From HEK293 Cells. Protein Expression And Purification, No 46, pp 321-331, 2006.
Petersen, N., Brunak, S., von Heijine, G. and H. Nielsen. SignalP 4.0: Discriminating Signal Peptides From Transmembrane Regions. Nature Methods, No 8, pp 785-786, 2011.
Myers, M. and L. M. Mulligan. The RET Receptor Is Linked To Stress Response Pathways. Cancer Research, No 64, pp 4453-4463, 2004.
Marchant, D. J., Bellac, C. L., Moraes, T. J., Wadsworth, S. J., Dufour, A., Butler, G. S., Bilawchuk, L. M., Hendry, R. G., Robertson, A. G., Cheung, C. T., Ng, J., Ang, L., Luo, Z., Heilbron, K., Norris, M. J., Duan, W., Bucyk, T., Karpov, A., Devel, L., Georgiadis, D., Hegele, R. G., Luo, H., Granville, D. J., Dive, V., McManus, B. M and Christopher M. Overall. a New Transcriptional Role For Matrix Metalloproteinase-12 In Antiviral Immunity. Nature Medicine, Vol. 20, No 5, pp 493-502, 2014.
G. A. Luke. Translating 2A Research Into Practice. Innovations in Biotechnology, 2012. InTech, Available from: www.intechopen.com
Kim, J. H., Lee, S-R., Li, L-H., Park, H-J., Park, J-H., Lee, K. Y., Kim, M.-K., Shin, B. A. and S-Y. Choi. High Cleavage Efficiency Of A 2A Peptide Derived From Porcine Teschovirus-1 In Human Cell Lines, Zebrafish And Mice. Plos ONE, Vol. 6, No 4, e18556, pp 1-8, 2011.

1. A nucleic acid expression vector for expressing a peptide of interest in a mammalian cell, the vector comprising a vector backbone;
the vector backbone comprising:
a first promoter for bacterial cells and a first selectable marker downstream of the first promoter;
a second promoter for eukaryotic cells and a second selectable marker downstream of the second promoter, wherein the second promoter is different from the first promoter and the second selectable marker is different from the first selectable marker;
nucleic acid encoding a signal peptide;
a multiple cloning site, for inserting a nucleic acid encoding the peptide of interest in-frame with the signal peptide;
a third promoter for expressing the nucleic acid encoding the peptide of interest; and
a 3′ UTR sequence comprising a polyadenylation sequence;
wherein the vector backbone consists of 4600 or fewer nucleic acid base pairs.

2. The nucleic acid expression vector according to claim 1, wherein the second promoter is weaker than an EF1A promoter.

3. The nucleic acid expression vector according to claim 1 further comprising the nucleic acid encoding the peptide of interest.

4. The nucleic acid expression vector according to claim 3 wherein the peptide of interest is a mammalian protein.

5. The nucleic acid expression vector according to claim 1 wherein the third promoter is a promoter for constitutive expression.

6. The nucleic acid expression vector according to claim 5 wherein the third promoter is a CMV promoter.

7. The nucleic acid expression vector according to claim 1 wherein the third promoter is a promoter for inducible expression.

8. The nucleic acid expression vector according to claim 7 wherein the third promoter is a doxycycline inducible promoter.

9. The nucleic acid expression vector according to claim 1 wherein the vector backbone further comprises a translational enhancer.

10. The nucleic acid expression vector according to claim 9 wherein the translational enhancer is SP163.

11. The nucleic acid expression vector according to claim 1 wherein the vector backbone further comprises nucleic acid encoding a SUMO-tag.

12. A host cell comprising the nucleic acid expression vector of claim 1.

13. The host cell according to claim 12, wherein the host cell is a mammalian cell.

14. The host cell according to claim 13, wherein the mammalian cell is selected from a HEK293 cell, a CHO cell, a COS cell, a HeLa cell, a Vero cell, a NSO cell, a Jurkat cell, a BHK cell, an MCF cell, or an L cell mouse fibroblast.

15. A nucleic acid expression vector comprising a vector backbone, the vector backbone comprising:
a first promoter for bacterial cells and a first selectable marker downstream of the first promoter;
a second promoter for eukaryotic cells and a second selectable marker downstream of the second promoter, wherein the second promoter is different from the first promoter and the second selectable marker is different from the first selectable marker;
a CMV promoter for expressing nucleic acid encoding a peptide of interest;
nucleic acid encoding a signal peptide downstream of the CMV promoter;
a multiple cloning site downstream of the nucleic acid encoding the signal peptide, for inserting the nucleic acid encoding the peptide of interest in-frame with the signal peptide; and
a polyadenylation sequence downstream of both the multiple cloning site and the second selectable marker;
wherein the second promoter is an SV40 promoter or a promoter that has weaker expression induction than an SV40 promoter; and
wherein the vector backbone consists of fewer than 4600 nucleic acid base pairs.

16. A method of making a nucleic acid expression vector, the method comprising introducing a nucleic acid that encodes a signal peptide into a plasmid that comprises:
a first promoter for bacterial cells and a first selectable marker downstream of the first promoter;
a second promoter for eukaryotic cells and a second selectable marker downstream of the second promoter, wherein the second promoter is different from the first promoter and the second selectable marker is different from the first selectable marker;
a multiple cloning site, for inserting nucleic acid encoding a peptide of interest in-frame with the signal peptide;
a third promoter for expressing the nucleic acid encoding the peptide of interest; and
a 3′ UTR sequence comprising a polyadenylation sequence;
wherein the resulting nucleic acid expression vector comprises a vector backbone consisting of 4600 or fewer nucleic acid base pairs.

17. A kit for gene expression comprising a vector according to claim 1, a mammalian cell, and a reagent.

18. A method for expressing a gene of interest, the method comprising:
inserting nucleic acid encoding the gene of interest into a nucleic acid expression vector according to claim 1;
transfecting the nucleic acid expression vector comprising the gene of interest into a mammalian cell; and
culturing the transfected cell.

19. The method according to claim 18 further comprising the step of purifying the protein encoded by the gene of interest.

20. Nucleic acid encoding modified EF1A 1 or EF1A2, the nucleic acid having at least 90% sequence identity to SEQ ID NO: 41 or SEQ ID NO: 42 or SEQ ID NO: 43.


SEQUENCE LISTING (48 sequences)

SEQ ID NO: 1 - protein, 17 aa, synthetic construct
  REGION 1..17: note = Gaussia luciferase signal peptide
MGVKVLFALICIAVAEA

SEQ ID NO: 2 - protein, 24 aa, synthetic construct
  REGION 1..24: note = PDX-Furin inhibitor signal peptide
MPSSVSWGILLLAGLCCLVPVSLA

SEQ ID NO: 3 - protein, 17 aa, synthetic construct
  REGION 1..17: note = BM40 signal peptide
MRAWIFFLLCLAGRALA

SEQ ID NO: 4 - DNA, 298 bp, synthetic construct
  misc_feature 1..298: note = DNA fragment 1
  source 1..298: mol_type = other DNA
gctgggtaccgaaattaatacgactcactatagggagacccaagctggcttgcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatgagggcctggatcttctttctcctttgcctggccgggagggctctggcagccccgctagccgag

SEQ ID NO: 5 - DNA, 301 bp, synthetic construct
  misc_feature 1..301: note = DNA fragment 2
  source 1..301: mol_type = other DNA
gctgggtaccgaaattaatacgactcactatagggagacccaagctggcttgcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatgggagtcaaagttctgtttgccctgatctgcatcgctgtggccgaggccaagcccacgctagccgag

SEQ ID NO: 6 - DNA, 116 bp, synthetic construct
  misc_feature 1..116: note = DNA fragment 3
  source 1..116: mol_type = other DNA
gctgggtaccgaaattaatacgactcactataggggaaccagccaccatgggagtcaaagttctgtttgccctgatctgcatcgctgtggccgaggccaagcccacgctagccgag

SEQ ID NO: 7 - DNA, 428 bp, synthetic construct
  misc_feature 1..428: note = F1ori sequence
  source 1..428: mol_type = other DNA
gcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatta

SEQ ID NO: 8 - DNA, 74 bp, synthetic construct
  misc_feature 1..74: note = EM7 sequence
  source 1..74: mol_type = other DNA
cagcacgtgttgacaattaatcatcggcatagtatatcggcatagtataatacgacaaggtgaggaactaaatc

SEQ ID NO: 9 - DNA, 1290 bp, synthetic construct
  misc_feature 1..1290: note = EF1a Genomic
  source 1..1290: mol_type = other DNA
aacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaaatgggaaag

SEQ ID NO: 10 - DNA, 342 bp, synthetic construct
  misc_feature 1..342: note = EF1A pPEF
  source 1..342: mol_type = other DNA
aacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaaaactacccctaaaagccaaaatgg

SEQ ID NO: 11 - DNA, 1217 bp, synthetic construct
  misc_feature 1..1217: note = EF1A commercial
  source 1..1217: mol_type = other DNA
agatctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaaagatct

SEQ ID NO: 12 - DNA, 1206 bp, synthetic construct
  misc_feature 1..1206: note = Fragment I with minimal EF1A
  source 1..1206: mol_type = other DNA
gctagccgaggttaactgttgtcctggctgttgcggttccggacaccatcatcaccaccatcaccatcaccattgaggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaaaactacccctaaaagcttcaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtg

SEQ ID NO: 13 - DNA, 1214 bp, synthetic construct
  misc_feature 1..1214: note = Fragment II with SV40 promoter
  source 1..1214: mol_type = other DNA
gctagccgaggttaactgttgtcctggctgttgcggttccggacaccatcatcaccaccatcaccatcaccattgaggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagcttaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtg

SEQ ID NO: 14 - protein, 315 aa, synthetic construct
  REGION 1..315: note = N-terminal SUUMOstar secretory tag
HHHHHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGQHYLHIRPAPSDNLPLVDLIEHPDPIFDPKEKDLNETLLRSLLGGHYDPGFMATSPPEDRPGGGGGPAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 15 - protein, 352 aa, synthetic construct
  REGION 1..352: note = Ricin-C3 P2A of PLA2R1
LAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTGSGHHHHHHHHHHRRKRGSGATNFSLLKQAGDVEENPGPQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPGSGHHHHHHHHHH

SEQ ID NO: 16 - DNA, 346 bp, synthetic construct
  misc_feature 1..346: note = pPEF5.2
  source 1..346: mol_type = other DNA
atataagcagagctcgtttagtgaaccgtcagatctctagaagctgggtaccgaaattaatacgactcactataggggaaccagccaccatgggagtcaaagttctgtttgccctgatctgcatcgctgtggccgaggccaagcccacgctagccgaggttgctttgaggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtccttt

SEQ ID NO: 17 - DNA, 4561 bp, synthetic construct
  misc_feature 1..4561: note = pPEF5.2
  source 1..4561: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgaaattaatacgactcactataggggaaccagccaccatgggagtcaaagttctgtttgccctgatctgcatcgctgtggccgaggccaagcccacgctagccgaggttgctttgaggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagcttaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 18 - DNA, 4746 bp, synthetic construct
  misc_feature 1..4746: note = pPEF5.2 max
  source 1..4746: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgaaattaatacgactcactatagggagacccaagctggcttgcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatgggagtcaaagttctgtttgccctgatctgcatcgctgtggccgaggccaagcccacgctagccgaggttgctttgaggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagcttaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 19 - DNA, 5223 bp, synthetic construct
  misc_feature 1..5223: note = pPEF1
  source 1..5223: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgaaattaatacgactcactatagggagacccaagctggcttgcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatgagggcctggatcttctttctcctttgcctggccgggagggctctggcagccccgctagccgagggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 20 - DNA, 5226 bp, synthetic construct
  misc_feature 1..5226: note = pPEF2
  source 1..5226: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgaaattaatacgactcactatagggagacccaagctggcttgcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatgggagtcaaagttctgtttgccctgatctgcatcgctgtggccgaggccaagcccacgctagccgagggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 21 - DNA, 5068 bp, synthetic construct
  misc_feature 1..5068: note = pPEF3
  source 1..5068: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccttaaggcgccagctgatcaagcttctgcctgccgcctgcctgcctgccactgagggttcccagcaccatgagggcctggatcttctttctcctttgcctggccgggagggctctggcagccccgctagccgagggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 22 - DNA, 5041 bp, synthetic construct
  misc_feature 1..5041: note = pPEF4
  source 1..5041: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgaaattaatacgactcactataggggaaccagccaccatgggagtcaaagttctgtttgccctgatctgcatcgctgtggccgaggccaagcccacgctagccgagggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 23 - DNA, 4553 bp, synthetic construct
  misc_feature 1..4553: note = pPEF5.1
  source 1..4553: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgaaattaatacgactcactataggggaaccagccaccatgggagtcaaagttctgtttgccctgatctgcatcgctgtggccgaggccaagcccacgctagccgaggttgctttgaggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaaaactacccctaaaagcttcaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 24 - DNA, 4738 bp, synthetic construct
  misc_feature 1..4738: note = pPEF5.1max
  source 1..4738: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgaaattaatacgactcactatagggagacccaagctggcttgcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatgggagtcaaagttctgtttgccctgatctgcatcgctgtggccgaggccaagcccacgctagccgaggttgctttgaggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaaaactacccctaaaagcttcaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 25 - DNA, 9901 bp, synthetic construct
  misc_feature 1..9901: note = pCEP-Pu/AC7
  source 1..9901: mol_type = other DNA
gccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcatgctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccttaaggcgccagctgatcaagcttctgcctgccgcctgcctgcctgccactgagggttcccagcaccatgagggcctggatcttctttctcctttgcctggccgggagggctctggcagccccgctagctctcactgaaacagatatatgcaagttgccgaaagacgaaggaacttgcagggatttcatattaaaatggtactatgatccaaacaccaaaagctgtgcaagattctggtatggaggttgtggtggaaacgaaaacaaatttggatcacagaaagaatgtgaaaaggtttgcgctcctgtgctcgccaaacccggagtcatcagtgtgatgggaacctaagcgtgggtggccaacatcatatacctcttgaagaagaaggagtcagccatcgccaacttgtctcgaggtccgcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgactctaga

SEQ ID NO: 26 - DNA, 163 bp, synthetic construct
  misc_feature 1..163: note = QBI SP163 translational enhancer
  source 1..163: mol_type = other DNA
agcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaacc

SEQ ID NO: 27 - DNA, 73 bp, synthetic construct
  misc_feature 1..73: note = Polylinker/MCS
  source 1..73: mol_type = other DNA
acgctagccgaggttaactgttgtcctggctgttgcggttccggacaccatcatcaccaccatcaccatcacc

SEQ ID NO: 28 - DNA, 77 bp, synthetic construct
  misc_feature 1..77: note = Polylinker/MCS
  source 1..77: mol_type = other DNA
attgaggatccagtgtggtggaattctgcagatatccagcacagtggcggccgctcgagtctagagggcccgtttaa

SEQ ID NO: 29 - DNA, 73 bp, synthetic construct
  misc_feature 1..73: note = Polylinker/MCS
  source 1..73: mol_type = other DNA
acgctagccgaggttgctttgaggatccagtgtggtggaattctgcagatatccagcacagtggcggccgctc

SEQ ID NO: 30 - DNA, 21 bp, synthetic construct
  misc_feature 1..21: note = 31
  source 1..21: mol_type = other DNA
gagtctagagggcccgtttaa

SEQ ID NO: 31 - DNA, 344 bp, synthetic construct
  misc_feature 1..344: note = SV40 promoter
  source 1..344: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 32 - DNA, 577 bp, synthetic construct
  misc_feature 1..577: note = CMV immediate early promoter
  source 1..577: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagc

SEQ ID NO: 33 - DNA, 342 bp, synthetic construct; identical to SEQ ID NO: 10
  misc_feature 1..342: note = EF1 alpha promoter minimal
  source 1..342: mol_type = other DNA

SEQ ID NO: 34 - DNA, 1285 bp, synthetic construct
  misc_feature 1..1285: note = EF1 alpha promoter genomic
  source 1..1285: mol_type = other DNA
aacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaaatgg

SEQ ID NO: 35 - DNA, 1217 bp, synthetic construct; identical to SEQ ID NO: 11
  misc_feature 1..1217: note = EF1 alpha promoter commercial
  source 1..1217: mol_type = other DNA

SEQ ID NO: 36 - DNA, 122 bp, synthetic construct
  misc_feature 1..122: note = Deletion fragment of MCS primer
  source 1..122: mol_type = other DNA
cgctgtggccgaggccaagcccacgctagccgaggttaactgttgtcctggctgttgcggttccggacaccatcatcaccaccatcaccatcaccattgaggatccagtgtggtggaattct

SEQ ID NO: 37 - DNA, 21 bp, synthetic construct
  misc_feature 1..21: note = pPEF5.2 reverse primer
  source 1..21: mol_type = other DNA
caacagccaggacaacagtta

SEQ ID NO: 38 - DNA, 30 bp, synthetic construct
  misc_feature 1..30: note = pPEF5.2 Forward primer introducing AfeI
  source 1..30: mol_type = other DNA
ggtgagcgctttgaggatccagtgtggtgg

SEQ ID NO: 39 - DNA, 901 bp, synthetic construct
  misc_feature 1..901: note = Synthetic PuroR DNA
  source 1..901: mol_type = other DNA
caaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagcttgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtg

SEQ ID NO: 40 - DNA, 4802 bp, synthetic construct
  misc_feature 1..4802: note = pPEF5PuroR
  source 1..4802: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgaaattaatacgactcactataggggaaccagccaccatgggagtcaaagttctgtttgccctgatctgcatcgctgtggccgaggccaagcccacgctagccgaggttgctttgaggatccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagcttgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgaaagtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 41 - DNA, 290 bp, synthetic construct
  misc_feature 1..290: note = Minimal EF1A1
  source 1..290: mol_type = other DNA
cctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtga

SEQ ID NO: 42 - DNA, 156 bp, synthetic construct
  misc_feature 1..156: note = Minimal EF1A2
  source 1..156: mol_type = other DNA
gcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtga

SEQ ID NO: 43 - DNA, 157 bp, synthetic construct
  misc_feature 1..157: note = Minimal EF1A3
  source 1..157: mol_type = other DNA
aacccagagatcgctgcgttcccgccccctcacccgcccgctctcgtcatcactgagcctttttcccgagggtgggggagaaccgtatataagtgcagtagtcgccgtgaacgttctttttcgcaacgggtttgccgccagaacacaggtgtcgtga

SEQ ID NO: 44 - DNA, 26 bp, synthetic construct
  misc_feature 1..26: note = Forward primer EF_Forward1
  source 1..26: mol_type = other DNA
tttaggcctcacccgcccgctctcgt

SEQ ID NO: 45 - DNA, 22 bp, synthetic construct
  misc_feature 1..22: note = Forward primer EF_Forward2
  source 1..22: mol_type = other DNA
tttaggcctagagaaggtggcg

SEQ ID NO: 46 - DNA, 30 bp, synthetic construct
  misc_feature 1..30: note = Reverse primer EF_Reverse1&2
  source 1..30: mol_type = other DNA
tttaggcctgcgtaaccaccacacccgccg

SEQ ID NO: 47 - DNA, 24 bp, synthetic construct
  misc_feature 1..24: note = Forward primer EF_Forward3
  source 1..24: mol_type = other DNA
tttaggcctttttcccgagggtgg

SEQ ID NO: 48 - DNA, 27 bp, synthetic construct
  misc_feature 1..27: note = Reverse primer EF_Reverse3
  source 1..27: mol_type = other DNA
tttaggcctcagtgatgacgagagcgg